(19) United States
(12) Patent Application Publication
(10) Pub. No.: US 2005/0037435 A1
(43) Pub. Date: Feb. 17, 2005

(54) Random chemistry for the generation of new compounds

(76) Inventors: Stuart A. Kauffman, Santa Fe, NM (US); Julius Rebek JR., Cambridge, MA (US)

Correspondence Address: HUTCHISON & MASON PLLC, PO box 31686, RALEIGH, NC, 27612, US

(21) Appl. No.: 10/933,083

(22) Filed: Sep. 2, 2004

Related U.S. Application Data
(63) Continuation of application No. 08/882,950, filed on Jun. 26, 1997.
Application No. 08/882,950 is a continuation of application No. 08/537,736, filed on Jan. 29, 1996, now abandoned.
Application No. 08/537,736 is a continuation-in-part of application No. 08/049,268, filed on Apr. 19, 1993, now abandoned.

(51) Int. Cl. G01N 33/53; G01N 33/543

(52) U.S. Cl. 435/7.1; 436/518

(57) ABSTRACT

Methods for the generation of new compounds are disclosed. The present invention eliminates the need to know in advance the structure or chemical composition of a compound having a desired property. The disclosure of the present invention provides that diversity of unknown compounds may be produced by “random” chemistry, and such a diversity of unknown compounds may be screened for one or more desired properties to detect the presence of suitable compounds. In one aspect, a starting group of organic compounds is caused to undergo a series of chemical reactions to create a diversity of new organic compounds that are screened for the presence of organic compounds having the desired property. In another aspect of the present invention, a diversity of compounds is generated from a group of substrates which are subjected to a group of enzymes representing a diversity of catalytic activities.

### TECHNICAL FIELD

The present invention relates generally to the generation of new compounds without predetermining a desired structure or composition, and the screening of such compounds for one or more desired properties. This invention is more particularly related to the use of a random chemistry, with or without enzymes, to generate a variety of new compounds from which those with a desired property may be characterized or identified, e.g., for subsequent production in batch quantities by conventional methodologies or otherwise.

### BACKGROUND OF THE INVENTION

Humankind's attempt to acquire new and useful compounds has been one of the more interesting but problematic endeavors, especially with respect to medically useful compounds. In general, the traditional approaches to the acquisition of new compounds have been either isolation of natural products (i.e., isolation of compounds found in nature) or synthetic preparation. Discovery of new and useful compounds via natural products is hampered by a variety of problems, including the availability of source materials from which to isolate compounds. Further, the variety of compounds via natural products is not unlimited, as plants and other living organisms do not make every compound theoretically possible.

Alternatively, new compounds have been prepared synthetically, i.e., by the creation of compounds in the laboratory rather than through the isolation of naturally occurring compounds. The synthetic generation of compounds utilizes the principles and methodologies of organic chemistry, especially reaction mechanisms. Compounds are created by deliberate, “rational” approaches in which the structure of a desired compound is first determined or conceived, and a synthesis strategy is then developed. This approach appears to be reaching its zenith in the field of drug design where computer-assisted, structure-activity studies are performed to generate rational drug design. However, in general, rational drug design has not achieved the successes initially envisioned.

Once a desired compound structure is identified, a strategy for synthetic preparation is developed. Traditional strategies for organic synthesis are serial synthesis or assembly of subunits in parallel, or a combination thereof. Serial synthesis involves the modification of one compound to form another compound, which in turn is chemically transformed (and so on), until the desired compound is synthesized. Assembly of subunits in parallel involves the synthesis of “portions” of a desired compound with production of the desired compound resulting from the joining of the individual portions.

More specifically, current techniques to synthesize desired compounds through a sequence of catalyzed reactions are based on the control of each reaction step in a sequential synthesis to optimize the yield of each intermediate compound used along the pathway of synthesis of the specific deed in product compound. The logic of this established procedure rests on the fact that the structure of the desired terminal product molecule is known beforehand, and that a thermodynamically efficient reaction pathway leading from substrates to the desired product exists. As noted above, two major general strategies to synthesize a desired target compound are common in the art. In the first, the terminal target is built up sequentially by successive modification of a starting substrate, acted upon in conjunction with other possible substrates, either by enzymes or careful choice of reaction conditions. A simple example is the sequential chemical synthesis of a desired peptide by cycles of protection and deprotection of the growing peptide chain as a succession of activated amino acids is added one by one. A second major alternative strategy in the art is the synthesis of a desired chemical compound by the successive synthesis of increasingly complex sets of building blocks which are finally joined to make the desired target. A simple example is the synthesis of a specific hexapeptide (ABCDEF) from the amino acid monomers A, B, C, D, E, F, by the synthesis of the dipeptides AB, CD, EF, then the joining of the dipeptides to form the hexapeptide. The same two general strategies are utilized in many areas of synthetic chemistry with a variety of different organic compounds. Both strategies are hindered by a variety of problems, including the necessity for knowledge about, and use of, prespecified reaction pathways.

In summary, current approaches to the acquisition of new and useful compounds are subject to a variety of limitations. Thus, there is a need in the art for a method for generating new compounds without the necessity for predetermining chemical structures, compositions, or synthesis pathways. The present invention fulfills these needs and further provides other related advantages.

### SUMMARY OF THE INVENTION

In contrast to the current approaches to the acquisition of new and useful compounds, the present invention eliminates the need to know the structure or chemical composition of the desired compound prior to its synthesis. The disclosure of the present invention provides that a diversity of unknown compounds may be produced by “random” chemistry, and such a diversity may be screened for one or more desired properties to detect the presence of suitable compounds. It is central to the subject methods that one does not need to know in advance the structure or composition of the useful compound sought.

Briefly stated, the present invention provides methods for the production of an organic molecule having a desired property, or for the generation and characterization of an organic molecule having a desired property.

In accordance with a first aspect of the present invention, the method comprises first providing a starting group of different organic molecules. At least one chemical reaction is caused to take place with at least some of the different organic molecules in the starting group to create an intermediate reaction mixture having one or more organic molecules different from the organic molecules in the starting group. This step of causing at least one chemical reaction to take place is repeated at least once. Each repetition uses the reaction mixture of the previous step, and in the end produces a final reaction mixture as a result of the last repetition. The final reaction mixture is screened for the presence of the organic molecule having the desired property.

In accordance with an embodiment of the first aspect, the method for the production of an organic molecule having a desired property as described above is performed. If the screening step of this aspect is successful in detecting the organic molecule having the desired property in the final reaction mixture, then the following additional steps are performed. The starting group of different organic molecules is divided into at least two subgroups, each containing less than all of the different organic molecules in the starting group. The chemical reactions are performed on each of the subgroups in the same way as with the starting group to produce a final reaction submixture corresponding to each of the subgroups. Each of the final reaction submixtures resulting from this step is screened for the presence of the organic molecule having the desired property. These additional steps are repeated at least once for each of the successful subgroups from which the organic molecule having the desired property is produced, by substituting the successful subgroup as the subgroup in the first additional step to thereby identify a narrowed group of different organic molecules from which the compound having the desired property can be produced.

In one embodiment, the method comprises the steps of:
(a) reacting a group of different substrates, the group comprising acids, amines, alcohols, and unsaturated compounds, under suitable conditions with a dehydrating agent to yield a first reaction mixture;
(b) reacting the first reaction mixture with a reducing agent under suitable conditions to yield a second reaction mixture;
(c) reacting the second reaction mixture with an oxidizing agent under suitable conditions to yield a third reaction mixture;
(d) performing a condensation reaction under suitable conditions upon the third reaction mixture to yield a fourth reaction mixture;
(e) exposing the fourth reaction mixture to light within a wavelength of about 220 nanometers to 600 nanometers, thereby producing one or more organic molecules different from the substrates and agents;
(f) screening the exposed fourth reaction mixture for the presence of an organic molecule having the desired property; and
(g) isolating from the exposed fourth reaction mixture the organic molecule having the desired property.

In an alternative embodiment, any subset of steps a-e above may be performed in any order prior to steps f and g. Further, steps a-e or any subset of these may be repeated in any order prior to steps f and g. Similarly, exposure to other reagents, singly, sequentially, or simultaneously, may be substituted for steps a-e, prior to steps e and f.

In another embodiment of the first aspect, the method comprises the steps of:
(a) reacting a group of different substrates, the group comprising acids, amines, alcohols, and unsaturated compounds, under suitable conditions with a dehydrating agent to yield a first reaction mixture;
(b) reacting the first reaction mixture with a reducing agent under suitable conditions to yield a second reaction mixture;
(c) reacting the second reaction mixture with an oxidizing agent under suitable conditions to yield a third reaction mixture;
(d) performing a condensation reaction under suitable conditions upon the third reaction mixture to yield a fourth reaction mixture;
(e) exposing the fourth reaction mixture to light within a wavelength of about 220 nanometers to 600 nanometers, thereby producing one or more organic molecules different from the substrates and agents;
(f) screening the exposed fourth reaction mixture for the presence of an organic molecule having the desired property; and
(g) determining the structure or functional properties characterizing the organic molecule having the desired property.

Any subset of steps a-e above may be performed in any order prior to steps f and g. Further, steps a-e or any subset of these may be repeated in any order prior to steps f and g. Similarly, exposure to other reagents, singly, sequentially, or simultaneously, may be substituted for steps a-e, prior to steps e and f.

In accordance with a second aspect of the present invention, the method comprises the steps of:
(a) reacting a group of different enzymes representing a diversity of catalytic activities under suitable conditions with a group of different substrates, thereby producing one or more organic molecules different from the enzymes and substrates in the reaction mixture;
(b) screening the reaction mixture for the presence of an organic molecule having a desired property; and
(c) isolating from the reaction mixture the organic molecule having the desired property.

In one embodiment of the second aspect, the method comprises the steps of:
(a) reacting a group of different enzymes representing a diversity of catalytic activities under suitable conditions with a group of different substrates, thereby producing one or more organic molecules different from the enzymes and substrates in the reaction mixture;
(b) screening the reaction mixture for the presence of an organic molecule having the desired property; and
(c) determining the structure or functional properties characterizing the organic molecule having the desired property.

Other aspects of the invention will become evident upon reference to the following detailed description.

### DETAILED DESCRIPTION OF THE INVENTION

In the first aspect of the present invention, the method comprises first providing a starting group of different organic molecules. At least one chemical reaction is caused to take place with at least some of the different organic molecules in the starting group to create an intermediate reaction mixture having one or more organic molecules different from the organic molecules in the starting group. This step of causing at least one chemical reaction to take place is repeated at least once. Each repetition uses the reaction mixture of the previous step, and in the end produces a final reaction mixture as a result of the last repetition. The final reaction mixture is screened for the presence of the organic molecule having the desired property.

As noted above, in another aspect, a diversity of compounds is generated from a group of substrates which are subjected to a group of enzymes representing a diversity of catalytic activities. In still another aspect of the present invention, a diversity of compounds is generated from a group of substrates which are subjected to a variety of conditions, in the absence of enzymes. An embodiment of either aspect utilizes a group of substrates with different core structures. Another embodiment of either aspect utilizes a group of substrates with similar or identical core structures, but a variety of different functional groups as substituents. The latter embodiment permits the creation of a diversity of compounds centered around a particular compound or a particular class of compounds.

The methods of the present invention are employed to generate new compounds having a desired property. Examples of preferred desired properties include the ability to function as drugs, vaccines, liganding agents, catalysts, catalytic cofactors, structures of use, detector molecules, and building blocks for other compounds. A liganding agent may bind, for example, to protein, DNA, RNA, carbohydrate, enzyme, receptor, or membrane. Liganding agents include agonists and antagonists, such as competitive inhibitors of enzymes or hormones. Structures of use include low energy structures (e.g., structures capable of self assembly) and material structures, like silk. Detector molecules include compounds having optical reporter properties of interest. A new compound may mimic, modulate, enhance, antagonize, modify, or simulate a substance. Specific molecules of interest include molecules: (1) able to bind to a helper T cell receptor of specific clones of helper T cells (e.g., such binding leads to amplification or deletion of specific helper T cell clones); (2) able to be incorporated into DNA or RNA in place of normal nucleotides (e.g., such incorporation alters biological activity); and (3) able to act as a substrate for an enzyme or modify the activity of an enzyme (e.g., may modify the binding activity of a biological molecule). Such molecules are useful for a variety of diagnostic and therapeutic purposes. Other specific molecules of interest include oral contraceptives and molecules with improved properties over analgesics like naproxen, or protease inhibitors like captopril, antitumor agents like mitomycin, antibiotics like vancomycin, and antifungals like amphotericin.

Substrates for the processes described herein include all organic compounds. A preferred group of substrates includes alkanes, alkenes, alkynes, arenes, alcohols, ethers, amines, aldehydes, ketones, acids, esters, amides, cyclic compounds, heterocyclic compounds, organometallic compounds, hetero-atom bearing compounds, amino acids, and nucleotides. A more preferred group of substrates includes acids, amines, alcohols, amino acids, nucleotides, and unsaturated compounds, such as alkenes and alkynes. The most preferred group of substrates is amino acid-based compounds (e.g., amino acids, peptides and polypeptides), nucleotide-based compounds (e.g., nucleotides and nucleosides), and combinations thereof. These substrates may include additional functional groups as substituents and may be acyclic, cyclic, and heterocyclic in nature. The acids, amines and alcohols can be primary, secondary, carboxylic, phosphoric, sulfonic, aromatic, heterocyclic, aliphatic, etc. For increased reactivity, primary amines and alcohols are preferred.

An alternative to the selection of different substrates with a wide variation in their overall structures is to choose substrates that include compounds which are different but share one or more common structural features with a molecule of interest or a class of molecules of interest. Thus, the diversity of compounds to be generated would be created around a molecule of interest or a class of molecules of interest. For example, a ringed compound, such as a steroid, may be selected and then a variety of different derivatives obtained. Derivatives include the addition and/or deletion of functional groups, and acyclic compounds with ringed substituents similar to a portion of the original cyclic compound. Such derivatives are subjected to the random chemistry processes described herein to generate a greater diversity, from which a compound having a desired property may be detected for further characterization, with or without isolation. For example, a group of substrates consists of related compounds, which are then subjected to the methods without enzymes as described herein. Alternatively, a group of substrates consists of related compounds plus reagents, which are then subjected to the methods with enzymes as described herein. A variation upon these embodiments of the present invention is to generate derivatives using the random chemistry processes described herein, and then subject such derivatives to these processes to generate a greater diversity, from which a compound having a desired property may be detected for further characterization, with or without isolation.

Classes of molecules, which are preferred focal points from which to obtain derivatives to serve as substrates, include heterocycles, steroids, alkaloids, and peptides/mimetics (including constrained molecules, e.g., constrained by. S—S disulfide bonds). Examples of heterocycles include purines, pyrimidines, benzodiazepins, beta-lactams, tetracyclines, cephalosporins, and carbohydrates. Examples of steroids include estrogens, androgens, cortisone, and ecdysone. Examples of alkaloids include ergots, vinca, curare, pyrollizidine, and mitomycines. Examples of peptides/mimetics include insulin, oxytocin, bradykinin, captopril, enalapril, and neurotoxins (e.g., from snails, snakes, etc.).

In one aspect, the present invention provides methods for generation of new compounds wherein a group of substrates are acted upon by a group of “enzymes,” such that a diversity of product molecules are formed. As used herein, the term “enzyme” includes enzymes (e.g., naturally or non-naturally occurring or produced), catalysts (e.g., catalytic surfaces), candidate catalysts and candidate enzymes (e.g., antibodies, RNA, DNA or random peptides/polypeptides). In one embodiment, the method comprises the steps of: (a) reacting a group of different enzymes representing a diversity of catalytic activities under suitable conditions with a group of different substrates, thereby producing one or more organic molecules different from the enzymes and substrates in the reaction mixture; (b) screening the reaction mixture for the presence of an organic molecule having a desired property; and (c) isolating from the reaction mixture the organic molecule having the desired property.

In another embodiment, the method comprises the steps of: (a) reacting a group of different enzymes representing a diversity of catalytic activities under suitable conditions with a group of different substrates, thereby producing one or more organic molecules different from enzymes and substrates in the reaction mixture; (b) screening the reaction mixture for the presence of an organic molecule having the desired property; and (c) determining the structure or functional properties characterizing the organic molecule have the desired property.

From a library of product molecules produced by the methods provided herein, those of practical interest are characterized. As noted above, it is central that, in the present procedures, one does not need to have prior knowledge of the structure or composition of the useful molecule sought. This aspect of the present invention rests on catalysis of, or otherwise causing, a sufficient diversity of reactions among a group of initial substrates, such that a diversity of further products are formed. In order to more fully appreciate the diversities of products which may be generated by the methods of the present invention, it may be helpful to consider a statistical analysis of the average properties of reaction graphs among a set of molecules, as well as the average properties of the catalyzed reaction subgraph among these molecules which is formed when the molecules are incubated in the presence of candidate enzymes or catalysts which may catalyze one or more of the reactions.

A reaction graph is the proper mathematical description of a set of organic molecules and all the reactions that those molecules can undergo. Organic reactions can be categorized into classes by the number of substrate and number of product molecule species. A first class transforms a single substrate into a single product. An isomerization reaction, catalyzed by an isomerase, is an example. A second class joins two substrates to form one product. A dehydration reaction joining two nucleotides by an ester bond, is an example. Such reactions are commonly catalyzed by ligases. A third broad class cleaves one substrate into two products. Cleavage of a polynucleotide by a phosphodiesterase is a familiar example, as are many steps in intermediate metabolism. Finally, a fourth class transforms two substrates into two products. Often this occurs by transfer of a reactive group from one of the two initial substrates to the second substrate.

A convenient representation of a reaction graph denotes each organic molecule species as a point in three dimensional space. One or two lines lead from the one or two substrate molecules derived from the reaction of the substrates. Arrows on the lines leaving the substrates point into a box denoting the reaction. Arrows leaving the reaction box for the products point toward the products. Since reactions are reversible, the arrows merely indicate one possible direction of the reaction. The set of all such arrows and boxes, representing all the reactions among all the organic molecules in the system, comprises the reaction graph.

An important feature of reaction graphs is that, for almost any initial set of organic molecules, the reaction graph in which that set is considered as substrates will also require addition of new organic molecules (i.e., molecules not in the initial set of substrates) where those new organic molecules are the products of one or more of the possible reactions among the initial set of substrates. In a mathematical process, called the “growth of the reaction graph”, the reaction graph “grows” by iterations. At the first step, a set of initial substrate molecules is listed. At the next step, the reaction graph among those substrates is formed mathematically, and a second iterate of the reaction graph is formed by listing both the initial substrates plus any new organic molecule products of the possible reactions. At the third step, all the possible organic reactions among this now enlarged set of organic molecules is written down. This new reaction graph may indicate that still further novel organic molecules are products of the reactions now possible. Over a succession of iterations of this mathematical graph growth process, the set of organic molecules included in the graph may increase enormously compared to the initial set of substrates. This successive increase is called “supracritical behavior.” Another possible mathematical behavior of the reaction graph growth processes is that a few new products may be formed on the first graph growth cycle, and successively fewer on the successive graph growth cycles, until no further new product molecules are generated. Behaviors in which graph growth is limited are termed “subcritical.”

If a set of organic molecules and a set of “enzymes,” as defined herein, are present in reaction conditions allowing the enzymes to act on the organic molecules, then the natural mathematical representation of the total system is the reaction graph, as defined above, plus an accounting of which enzymes catalyze which reactions. The latter accounting of enzymes and the reactions catalyzed comprises the catalyzed reaction subgraph of the reaction graph. This mathematic representation is formed by noting, for each candidate enzyme, which reactions if any it catalyzes. An arrow may then be drawn from that enzyme to the reaction box representing the reaction catalyzed, and the arrows into and out of the box representing transformations of substrate(s) into product(s) can be noted in a convenient way, e.g., by coloring those arrows “red.” The set of all red arrows represents the reactions which are catalyzed by one or more of the candidate enzymes present in the system.

Just as the reaction graph itself may be subcritical or supracritical in its behavior, so too may the catalyzed reaction subgraph among the organic molecules. In this case, one considers only the catalyzed reactions among the initial “founder” substrates. The catalyzed reactions lead to products not in the founder set of substrates. These new products are available, together with the initial founder set of substrates, to allow further reactions, some of which might be catalyzed by the set of candidate enzymes present in the system. Over a succession of iterations, this process of catalyzed reaction growth may increase vastly in diversity, in a supracritical mode. Alternatively, the set of novel molecules formed via catalyzed reactions may dwindle over successive iterations of the growth of the catalyzed reaction graph. This is a form of subcritical behavior.

The total behavior of the system is represented by the behavior of the reaction graph plus the catalyzed reaction subgraph, over iterations. The uncatalyzed reactions represent reactions that occur spontaneously. Whether a reaction graph behaves subcritically or supracritically depends upon the diversity of founder substrates and the diversity of candidate enzymes present in the system. In addition, the behavior is dependent upon factors including concentrations of all reagents, solubility of the organic molecules, and directions of deviation from equilibrium across each reaction.

In general, a phase transition from subcritical to supracritical behavior of a reaction system is governed by the diversity of organic molecules and the diversity of enzymes in the system. Systems with low diversity of both organic molecules and enzymes are typically subcritical. Systems with high diversities of either organic molecules alone, low diversities of organic compounds together with high diversities of enzymes, or high diversities of both are typically supracritical. Systems of organic molecules alone without addition of exogenous enzymes can be supracritical, because the spontaneous reaction graph is supracritical, or because some of the substrates or products are enzymes themselves in the sense defined above. In one of its forms, the present invention takes advantage of the mathematical phase transition between subcritical and supracritical behavior to choose reaction conditions which yield high diversity libraries of organic molecules from a founder set of organic molecules.

The general character of the phase transition from subcritical to supracritical behavior can be illustrated, by way of a non-limiting example, based on the preferred use of a cloned library of antibody molecules as the candidate set of enzymes, and a set of substrates which, without loss of generality, can be taken to be peptides containing mixtures of D and L amino acids and nonnatural amino acids, or can be taken to be small polynucleotides, or a wide variety of other organic molecules. To illustrate the general character of the phase transition it is useful to estimate the number of reactions in a reaction graph with a given number of small organic molecules. In general, the number of reactions is not known. However, minimum realistic estimates are obtainable. For example, a founder set of peptides made of D and L amino acids and non-natural amino acids, each with 10 amino acids, may be used as substrates. The number of possible substrates is very large, and given by the number of kinds of amino acids raised to the tenth power. Any two peptides length ten can undergo transpeptidation reactions cleaving and exchanging the terminal amino acid(s) subsequences at any of the internal peptide bonds of each of the decapeptides. Since there are 9 internal bonds in each, any pair of decapeptides can undergo 81 such transpeptidation reactions. In each case, two substrates yield two products. Since any pair of decapeptides can undergo 81 transpeptidation reactions, it is clearly an underestimate to suppose that the two peptides can undergo only 1 transpeptidation reaction. But even with this clear underestimate, the number of reactions in a system with a diversity of N types of peptides is equal to the number of possible pairs of peptides, hence equal to N squared. The same general features occur with many classes of organic molecules undergoing reactions with two substrates and two products. For most pairs of organic molecules, it is conservative to estimate that the two can undergo at least one reaction to form two products. Thus, in general, N squared is a conservative estimate of the diversity of reactions in a reaction graph with N kinds of organic molecules.

Again, as a non-limiting example to illustrate the general character of the phase transition in catalyzed reaction graphs, a set of 100,000,000 cloned human antibody molecules is used as the set of candidate enzymes. Based on the statistics of generating catalytic antibodies (as described below), the probability that a randomly chosen antibody molecule is able to catalyze a randomly chosen reaction is between 10⁻⁵ and 10⁻⁸ (Pollack et al., Science 234:1570, 1986; Tramontano et al., Science 234:1566, 1986; Tramontano et al., Proc. Natl. Acad. Sci. USA 83:6736, 1986; Jacobs et al., J. Am. Chem. Soc. 109:2174, 1987; Pollack and Schultz, Cold Spring Harbor Symposium on Quantitative Biology Vol. 52, 1987; Tramontano et al., Cold Spring Harbor Symposium on Quantitative Biology Vol. 52, 1987). The more conservative estimate, 10⁻⁸, may be used for illustration. Reaction systems in which the diversity of substrate molecules is varied are considered, and this diversity noted on the Y axis of the Cartesian coordinate system. Simultaneously, the diversity of the candidate set of enzymes is varied and noted on the X axis. Low diversity of substrates and candidate enzymes almost certainly yields subcritical reaction systems. To be concrete, a system with two substrates, hence one reaction, and a single randomly chosen antibody molecule is considered. The chance that this antibody molecule acts as a catalyst for any of the four single reactions afforded by the substrates is 10⁻⁸. Thus, almost certainly, no catalyst for the reaction is present in the system, and the formation of no novel product is catalyzed. The system is subcritical. A high diversity of substrates and candidate enzymes will be supracritical with high probability. A diversity of 1,000 organic molecules is incubated with a diversity of 1,000,000 antibody molecules in an appropriate reaction vessel. The number of reactions among the 1,000 organic molecules is at least, by the conservative estimate, 1,000,000. Each reaction might be catalyzed by any one of the 1,000,000 candidate antibody enzymes, and each antibody has a chance of one in a hundred million of being able to act as a catalyst for each reaction. Thus, the expected number of reactions for which antibody catalysts are present in the system is 10⁶×10⁶/10⁸=10⁴. Thus, 10,000 reactions among the million possible should be catalyzed by one or more of the antibodies present. Therefore, as these catalyzed reactions occur, the products of the 10,000 reactions will be formed. Most of these will differ from the 1,000 substrate molecules initially present. Thus, the diversity of the set of organic molecules has increased. After sufficient time has elapsed for the concentrations of these novel molecules to increase sufficiently, the new system has a diversity of substrates on the order of 10,000 rather than 1,000, hence, now a diversity of 10,000 squared reactions are possible among the enlarged set of substrates. The expected number of reactions which now find catalysts among the antibody molecules is thus 10⁸×10⁶/10⁸ or 1,000,000. Thus, within two reaction steps of the founder set of 1,000 organic molecules, the diversity of organic molecules has increased to about 1,000,000. Over successive reaction cycles, diversity will increase. This is supracritical behavior.

In general, in the X-Y plane, a roughly hyperbolic curve separates a subcritical regime near the origin, representing low diversities of founder substrates and enzymes, and a supracritical regime with high diversity of initial substrates, enzymes or both. With a fixed low diversity of substrates, the system can cross into the supracritical regime if a high enough diversity of enzymes is present. Conversely, if the enzyme diversity is fixed rather low, the system can cross into the supracritical regime if a high enough diversity of substrates is present. The actual shape of this roughly hyperbolic curve depends upon the specific way the number of reactions increases as substrate diversity increases, which in turn depends upon the particular set of organic molecules used as founder substrates. The curve also depends upon the distribution of probabilities that antibody molecules catalyze the different reactions afforded by the founder substrates and their products. However, for all these cases, a sufficient diversity of both substrates and enzymes leads to supracritical behavior. The diversity of organic molecules in the system will increase dramatically via the catalysis of connected webs of reactions leading from the founder set of organic molecules to an increasing diversity of their products.

It is important to emphasize that a system of substrates alone can explode into a diversity of products, even in the absence of exogenously supplied enzymes, if the substrates are present in sufficient diversity and high enough concentrations to interact on a reasonable time scale. For example, a large diversity will be generated if the spontaneous reaction graph is supracritical. However, systems with exogenously added enzymes are preferred.

The human antibody repertoire is used herein as a non-limiting example of a set of candidate enzyme molecules. As is known in the art, the combinatorial diversity of human antibody molecules due to genomic rearrangement is on the order of 100,000,000 (prior to the onset of somatic mutation during maturation of the immune response which further increases potential diversity). As is also known in the art, antibody molecules can function to catalyze a wide variety of reactions with a rate (Vmax) acceleration of three to eight orders of magnitude compared to the spontaneous reaction. Such catalytic antibodies are commonly generated by immunization of an immune competent animal with a molecule that is a stable analogue of the transition state of the desired reaction. Monoclonal antibodies are generated from this immunization, and each is tested for its capacity to catalyze the desired reaction. Because the stable analogue is similar chemically to the transition state of the reaction, typically on the order of 5% to 10% of the monoclonal antibodies tried are able to catalyze the desired reaction. Presumably the catalysis reflects high affinity for the transition state and lower affinity for substrates and products.

It is possible to estimate the probability that a randomly chosen antibody molecule will be able to catalyze a given, randomly chosen reaction. The fraction of B cells which respond to immunization with an arbitrary epitope bearing antigen is on the order of one in a hundred thousand. B cells which respond to an antigen typically have modestly high affinities for the antigen to be triggered to divide. Thus, the probability that a randomly chosen antibody molecule can bind with modest affinity, 10⁴ M⁻¹, to an arbitrary antigen is about one in a hundred thousand. The monoclonal antibodies used to create catalytic antibodies may have undergone further somatic mutation that increased affinity for the antigen. It is reasonable to estimate the probability that a randomly chosen antibody has high affinity for an arbitrary antigen is about 10⁻⁶ to 10⁻⁸.

It is further well known in the art that a cloned high diversity library (10⁸ or more) of antibody molecules can be and has been created in a variety of ways. Thus, such antibody libraries are a non-limiting example of a high diversity set of candidate enzymes.

The use of a repertoire of human antibodies as a set of candidate enzymes is a preferred, but non-limiting example of the sets of molecules which can advantageously be used as sets of enzymes. Additional candidate sets include the following:
(1) Libraries of fully random or partially stochastic polynucleotide sequences, DNA or RNA, which, upon translation yield libraries of fully or partially stochastic peptides, polypeptides or proteins. These libraries can be cloned in prokaryotic or eukaryotic hosts to amplify the polynucleotide sequences and obtain protein products which constitute the candidate enzyme library. Alternatively, the polynucleotide sequences can be amplified in vitro and translated in vitro to obtain the candidate enzyme library. If needed, the candidate protein library can be isolated from other molecular components by means known in the art. For example, an advantageous means to do so uses libraries of fusion proteins with stochastic peptides, polypeptides, or proteins fused adjacent to, for example, ubiquitin. Antibodies to ubiquitin allow affinity purification of the library of fusion proteins which then serves as the set of candidate enzymes.

(2) A library of antibody molecules can be derivatized by cloning partially stochastic DNA sequences into the hypervariable region of the antibody molecules. A refinement of this involves cloning such stochastic sequences into one or more of the complement determining regions (CDRs), of the antibody molecule. Each CDR has on the order of 5 to 10 amino acids. This modified library is a set of candidate enzymes.

(3) Partially stochastic DNA sequences or RNA sequences can be cloned into a gene encoding any protein, e.g., histone 1 or any other protein, to create a fusion protein with the novel DNA or RNA at one end, or in the middle of the host protein sequence. The well folded host protein serves as a framework to aid folding and stability of the cloned sequences. The set of such proteins is a library of candidate enzymes.

(4) Libraries of DNA sequences in themselves, or RNA sequences in themselves, constitute libraries of candidate enzymes. The existence of ribozymes and of DNA sequences able to bind arbitrary ligands, such as thrombin, show that both kinds of polymers are strong candidates to bind transition states and catalyze reactions.

(5) Other libraries of combinatorial molecular diversity, linear sequences or otherwise, as known in the art, may be used as candidate catalysts. Sets of known enzymes alone, or together with a small or large variety of mutant variants of those enzymes, can serve advantageously as the candidate set of enzymes. More specifically, and as a non-limiting example, the substrates of interest in generating a library of molecules may be D and L amino acids, including nonnatural amino acids, and some small dipeptides, tripeptides, and tetrapeptides formed of these building blocks. It is known in the art that larger peptides can be synthesized from amino acids and small peptides using proteases, peptidases, lipases, hydrolases, and esterases (Schellenberger and Jakubke, Chem. Int. Ed. Engl. 30:1437, 1991). Thus, such a set of enzymes can be used jointly in a common milieu, or sequentially, acting on a set of substrates. Further, it is known in the art that it is possible to select mutant variants of proteases which are able to alter substrate specificity, or alter catalytic activity in unusual solvents, such as low water dimethylformamide solvents (Arnold, Proc. Natl. Acad. Sci USA, in press 1993). Thus, in a preferred embodiment of the present invention, libraries of mutants of each of a set of enzymes of interest are used. For each enzyme, length N, there are 19N one mutuant variants, and on the order of that number squared of two mutant variants. Hence, a library of several million mutant proteins of a given enzyme, obtained by means known in the art, can be readily prepared. For a diversity of ten different initial enzymes, lipases, hydrolases, esterases, and proteases, the resulting library of candidate enzymes has on the order of 100,000,000 different protein species, each a candidate enzyme. These are then incubated with the founder substrate library of interest. Increase of the diversity of candidate proteins from 1,000,000 (described below in a non-limiting example based on antibody molecules) to 100,000,000, together with a maximum 10 mg/ml solubility of these proteins, implies that product molecules will form more slowly. Hence in a 1,000 microliter volume, it would require about 1 second to generate a 1 nanomolar concentration of a product molecule from saturated enzymes using a diversity of 1,000,000 candidate enzymes, and about 100-fold longer using a library of 100,000,000 candidate enzymes. The example of small D and L peptides is non-limiting. Other core building blocks, carbohydrates, heterocyclic compounds, a variety of adducts, and otherwise, can be used as the starting library of substrates in all the methods of the invention.

Where traditional protein-based enzymes are used to effect a diversity of catalytic activities, such enzymes include oxidoreductases; transferases; hydrolases; lyases; isomerases; and ligases. Oxidoreductases catalyze oxidation and reduction reactions. Examples of oxidoreductases include dehydrogenases; reductases; oxidases (monooxygenases and dioxygenases); and peroxidases. Transferases catalyze the transfer of functional groups. Examples of transferases include aminotransferases (transaminases); phosphotransferases; pyrophosphokinases; and nucleotidyltransferases (RNA and DNA polymerases). Hydrolases catalyze the hydrolytic cleavage of bonds, such as ester, glycosyl, and peptide bonds. Examples of hydrolases include phosphodiesterases; amylases; proteases (peptidases, proteinases); nucleases (exo- and endo-; ribo- and deoxyribonucleases); and phosphatases. Lyases catalyze double bond formation by non-hydrolytic removal of groups from substrates. Examples of lyases include decarboxylases; anhydrases; and synthases. Isomerases catalyze geometric or structural changes within one molecule. Examples of isomerases include racemases; epimerases; tautomerases; and mutases. Lyases catalyze the joining together of two molecules coupled with the hydrolysis of pyrophosphate bond. Examples of ligases include synthetases.

Generation of useful high diversity libraries requires that the substrates be soluble in the solvent, that the candidate enzymes be soluble in the solvent, that the volume be sufficiently small and concentrations sufficiently high that substrates and enzymes encounter one another rapidly, and at high enough concentrations to occupy a sufficient fraction of enzymatic sites to enhance reaction velocities, and that the high diversity product library be present in high enough concentrations that useful molecules can be detected. All these requirements have been considered for the present invention. For example, enzymes typically can tolerate some percentage of organic solvents such as ethanol, methanol, dimethyl sulfoxide (DMSO), dimethylformamide (DMF), or combinations thereof, in aqueous (water based) solutions (Gupia, Eur. J. Biochem. 205:25, 1991). Thus, where not all the substrates are water soluble, it is desirable to include water-miscible organic solvents.

Substrates of the types indicated vary in solubility. In general, it is reasonable to obtain millimolar concentrations of on the order of 1,000 substrate species in small reaction volumes, on the order of 1 to 100 microliters. Under reaction conditions such that the diversity of these 1,000 substrates increases by a factor of 1,000,000, yielding 1,000,000,000, or a library of small molecules with a diversity of 10⁹, the average concentration will have fallen by a factor of 10⁻⁶, hence have fallen from millimolar to nanomolar, 10⁻⁹ M. The detection methodologies discussed below to identify a molecule of interest are able to detect readily in the nanomolar range, and typically are able to detect in the picomolar, 10⁻¹² M, range. Thus, even with a 1,000-fold decrease in concentrations of some products below the mean when diversity is one billion, the detection means can detect molecules of interest. Other detection procedures allow detection at 10⁻¹⁵ to 10⁻²⁰ molar.

The diversity of candidate enzymes in a reaction mixture is limited by the solubility of the enzymes. For example, for proteins in aqueous media, a 10 mg/ml concentration is typically attainable. For candidate enzymes with 200 amino acids, on the order of 10²⁰ protein molecules can be in solution in 1,000 microliters. Thus, if a diversity of 10⁶ candidate enzymes is used, each will be present in 10¹⁴ copies. Catalytic antibodies are of modest efficiency, as noted. Using a turnover number of 1 per second, 10¹⁴ saturated enzymes would yield 10¹⁴ product molecules in 1 second. In a 1,000 microliter volume the concentration of the 10¹⁴ product molecules would be on the order of 0.1 micromolar. Even if solubility limits were 1 mg/ml of enzymes, then concentrations would decrease by only one order of magnitude. Thus, high diversities of substrates and candidate enzymes can be mixed under reaction conditions which yield a high diversity of products via a catalyzed web of reactions on practical time scales.

In an embodiment of this aspect of the present invention, a group of enzymes representing a diversity of catalytic activity are separated in part or in entirety from one another and the substrates contacted sequentially. For example, a group of enzymes are separated by membranes, such as dialysis bags, or by immobilizing different enzymes (representing different catalytic activities) on solid supports, such as resins. Candidate enzymes can be localized on phage, using phage display libraries as is known in the art, or other means to generate and display combinatorially diverse libraries of molecules, such as peptides or other molecules on beads, or surfaces, or polysome trapped peptide libraries. Additionally, candidate enzymes may be contained within or displayed upon one or more types of eukaryotic or prokaryotic cells, the cells and the substrates being brought into contact In any case, a group of substrates is circulated (e.g., by peristaltic pump) through the separated enzymes. For example, substrates are circulated in and out of dialysis bags with pore sizes which prevent escape of the enzymes. Substrates are bound by the first set of enzymes, modified, released and circulated to the next set of enzymes. Alternatively, a group of substrates may be confined and enzymes having one or more catalytic activities circulated through sequentially. In general, the reactions are conducted over a period of several hours at temperatures of about 37° C. or below. Cofactors such as ATP, NADH, O₂ and CoA are added where appropriate. Many of the cofactors may either be added directly or generated in situ. For example, O₂ may be introduced by injecting the gas or air directly into the reaction mixture or by use of an electrode to generate O₂. An electrode need not directly contact a reaction mixture, but rather may be introduced into a compartment from which O₂ may pass to the reaction mixture. For example, an electrode may be placed inside of a dialysis bag which in turn is surrounded by a dialysis bag containing a set of enzymes. It will be readily appreciated by those of ordinary skill in the art that a group of substrates may be subjected to the various separated enzymes in a variety of orders. Further, it will be evident that after subjecting a group of substrates to the various separated enzymes, one or more steps may be repeated if desired. The repetition of steps need not be in the order initially performed and additional substrates may be introduced at any step if desired.

In another embodiment of the present invention, a combinatorial library of organic molecules or other molecules, which are similar to an initial molecule of interest, are generated by derivatization of the initial molecule in a very large number of possible ways to produce a high diversity library of “local” mimics of the initial molecule of interest. Within the present invention, two ways are provided for generating such a library, one which does not use enzymes, but uses a variety of possible adducts or other molecules which may undergo reactions with the initial molecule of interest, and also uses a variety of chemical reagents and physical conditions to drive the synthesis of a library of derivatized products of the initial molecule. Alternatively, the core initial molecule plus a set of candidate adducts and other molecules which may react with the initial molecule are used, but also included is a set of enzymes which may increase the rate of formation of the local high diversity library of derivatized forms of the initial compound. Based upon the present disclosure provided herein, it will be readily appreciated by those of ordinary skill in the art that the methods for producing general high diversity libraries of product molecules and for producing local high diversity libraries of derivatized forms of an initial compound may be combined. For example, a new initial compound may be generated by the general procedure (e.g., substrates with different core structures). Such a new compound is then used, with or without derivatives, to generate a local high diversity library of derivatized forms of the compound. Further, it will be evident to those of ordinary skill in the art that libraries may be generated using a combination of the methods herein without enzymes and the methods herein with enzymes.

Generation of a high diversity library of derivatized forms of a steroid hormone core, such as estrogen, is used as a non-limiting example. A set of reactants, including estrogen and a variety of other small molecules which are candidates to react with estrogen to form new product molecules partially or entirely containing the steroid core, are utilized in a common reaction milieu. These are reacted in the presence of a set of enzymes to catalyze the reactions afforded by the system. Enzymes can be chosen by a number of means, some known in the art, others specified herein. The formation of a library of derivatized molecules under these reaction conditions can be assessed by a number of means known in the art. For example, the steroid core may be radioactively labeled at a variety of positions. Thereafter, the reaction mixture can be subjected to HPLC analysis, mass spectrograph analysis, or other modes of analysis to test for the diversity of molecules which are labeled. All radioactively labeled molecules contain atoms derived from the steroid core, hence the new molecule species are at least partially comprised of the steroid core. If it is desirable to assure that a large part of the steroid core is contained in the novel species, then two or more distinct radioactive labels can be used to label distinct and distant atoms in the core. Simultaneous presence of all labels suggests strongly that those portions of the steroid core are intact. Alternatives to radioactive labels include isotope labels and other means known in the art. The high diversity library is tested (e.g., by means described herein) to determine if it contains molecules of interest. If such molecules are detected, they may then be isolated by a variety of means, including sib selection as described herein.

The detection of molecules which are candidates to act as antagonists of estrogen is discussed first as a non-limiting example of detection of one or more molecules of interest in the library of this estrogen example. Detection of molecules which have higher affinity than estrogen for the estrogen receptor (and hence which may be of use in hormone replacement therapy at lower concentrations and thus lower side effects than estrogen itself) is discussed as a second non-limiting example. Detection of candidate antagonists in the reaction mixture may be accomplished by use of very high specific activity radioactive estrogen bound to receptors by means known in the art. Unlabeled competitors in the library will displace the labeled estrogen, and this competitive interaction can be detected by loss of label.

Detection of candidate high affinity agonists for replacement therapy may be carried out by use of appropriate cell assays similar to the frog melanocyte assay or the use of pH changes described in detail herein. Presence of a high affinity agonist in the reaction mixture is demonstrated because a very low concentration of the agonist compared to estrogen suffices to trigger the cell response. Such assays may be carried out in the absence of estrogen, or in the presence of increasing concentrations of estrogen. In the latter case, cell response at lower concentrations of estrogen than would elicit a response with estrogen alone, detects the presence of an agonist in the high diversity library. If the agonist can act alone to trigger the cell response, then during the sib selection winnowing procedure, as its concentration increases the threshold level of estrogen required for triggering a cell response will dwindle.

The creation of a set of candidate enzymes able to catalyze reactions derivatizing the core molecule, e.g., estrogen, is carried out by selecting from a large set of enzymes (for example the mouse or human immune repertoire), a subset of candidates which bind to the initial set of substrates, the core molecule plus the candidate substrates which are to react with the core and derivatize it. This set of enzymes may then advantageously be enlarged by generating a mutant variant spectrum of each. The purpose of this step is the following: The enzymes have been selected because they bind to the substrates of the potential reactions, rather than selectively binding the transition states of the reaction. Generation of mutant spectra around each such initial enzyme which binds substrate(s) increases the probability that the mixture of enzymes will include candidates which bind the transition state of the reaction, hence are improved candidates to catalyze the reaction.

In a repetitive procedure, a succession of candidate enzymes can advantageously be selected as candidates to catalyze the succession of reactions steps leading away from core molecule, for example the steroid core, and the initial adducts, to generate the high diversity library. At each reaction cycle with a given set of molecule, an enlarged set of molecules, many derivatized forms of the core molecule, will be generated. In order to find further enzymes to catalyze the next reactions afforded by the enriched reaction system to create still further derivatized molecules of the core, it is advantageous to select from a high diversity library of candidate enzymes, new candidates which may act on the newly formed species of product molecules. These new candidate enzymes plus their mutant spectra, as well as the previously identified candidate enzymes, may be used in the subsequent reaction cycle to catalyze the formation of still more kinds of derivatized forms of the core molecule. Given limited enzyme solubility, in order to keep the concentrations of critical enzymes as high as possible, it can be advantageous to utilize only the newly identified candidate enzymes, plus their mutant spectra identified from the high diversity library of candidate enzymes, plus the set of candidate enzymes from the last cycle or few cycles of the reaction sequence leading from the core molecule and initial adducts. In contrast, candidate enzymes leading from the initial core and initial adducts, can be advantageously eliminated in later iterative steps, since they have already acted to catalyze formation of their products.

For example, one means to identify such further enzyme candidates at each iterative step consists in labeling the substrate and the product molecules in the reaction mixture, each at a variety of positions, with radioactive iodine. The purpose of labeling a variety of positions on each compound with iodine is to assure that the iodine labeling of at least some members of that species of compounds will not prevent binding of candidate enzyme molecules at almost any compound site unhindered by the iodine label. These labeled molecules are then reacted with the high diversity of candidate library enzymes, for example with human antibody molecules, to detect which antibody molecules bind the labeled molecules from the reaction mixture. This set includes antibody molecules which bind the novel product molecules created in the reaction system. The antibody molecules plus their mutant variants are then used to enlarge the set of candidate enzymes.

A variety of means are known in the art to identify the antibody molecules which bind iodine labeled molecules in the reaction mixture. Among these, it is advantageous to use plaque assays or cell assays expressing the antibody library to test which plaques or cells bind iodine labeled material. If a fluorescent label is used instead of iodine, it is advantageous to make use of the natural display of antibody molecules on cell surfaces of immortalized B cells, where each such monoclonal antibody producing cell displays its unique antibody. It is then advantageous to expose the population of cells to the fluorescent labeled molecules in the reaction mixture, then sort the B cells. Those immortalized cells which are labeled generate antibody molecules which bind the labeled molecules from the reaction mixture. These immortalized cells can be grown to create a library of monoclonal antibodies which are the candidate enzymes. In addition, it is possible to select antibodies, or other sequences which constitute the further enzymes at each iterative step alluded to above by using the product molecules to create affinity columns, then using the columns to select subsets of libraries of phage displayed antibody molecules, polysome trapped antibodies, or libraries of DNA or RNA aptomers, or other sequences which bind the products on the column hence which may function as candidate enzymes. Thus, in addition to the use of a high diversity antibody library to find candidate enzymes, it is also possible to use other high diversity libraries. Among these, it is preferred to use high diversity RNA libraries, DNA libraries, and libraries of stochastic peptides alone or as fusion proteins with a variety of evolved proteins.

Another preferred means to create a set of candidate enzymes which may help derivatize a core molecule with a set of adducts or other substrates, consists in using known enzymes involved in the normal biosynthetic pathway leading to the core, plus mutant variants of those enzymes. Similarly, known enzymes utilizing any of the adducts as substrates, plus mutant variants of those enzymes, may be used. In order to catalyze a succession of reactions from the core molecule and further novel substrates which may react with it, it is advantageous to use the substrates and products present at each iteration of the reaction cycle to identify the enzymes which bind substrates and/or products, then create further mutant spectra of these identified enzymes as candidates to catalyze the next reaction steps from the core molecule. Enzymes which bind substrates and products can be identified by means known in the art, including binding assays to cloned enzymes via plaque or other assays. It is also advantageous to use a set of candidate enzymes formed by the union of a set of known enzymes and their mutant spectra, as just described, plus a set of candidates derived from a high diversity library of candidates, such as the mouse antibody repertoire as described above.

In all the embodiments of this invention it can be advantageous to use procedures to select substrates at each of the stages of amplification of diversity which are good candidates to undergo reactions which yield a desired molecule of interest. A procedure to do so consists in creating sets of “shape-complements” to the “shape” of the desired target, then using the sets of shape-complements to bind and affinity select candidate substrates whose own “shapes” are similar to the target shape of the desired molecule. As a non-limiting example, if the target molecule of interest is estrogen, it may be used to generate a set of monoclonal antibodies against estrogen, or a polyclonal serum against estrogen. These antibodies can be used to affinity purify candidate substrates with shapes similar to estrogen. Reactions building upon these candidate substrates can be carried out, and the products searched for estrogen mimics.

In addition to antibodies, other shape diversity libraries of DNA, RNA, or otherwise can be used to find shape-complements to the target molecule, here estrogen.

This “target shape” procedure can be advantageously extended in three ways. First, among the antibody molecules binding to estrogen (“rank one” antibodies), some will bind to the active site or the vicinity of the active site, and others will bind to other sites. These may be discriminated by using the antibodies, each as a monoclonal, to generate antiidiotype antibodies (“rank two” antibodies) by means known in the art. Any rank one antibody which generates a rank two aniidiotype antibody that competes with estrogen for the binding site on the rank one antibody is likely to be a rank one antibody whose binding site actually binds the active site of estrogen. The set of each such rank one antibodies can be used to affinity select candidate substrates with shapes similar to estrogen.

Second, the set of second rank antigens which compete with estrogen for binding sites on rank one antibodies can be used to affinity select candidate enzymes which will act on estrogen-like substrates to yield estrogen mimics.

Third, this set of rank two antibodies can be used to generate “rank three” antibodies which can be used to affinity select a wide variety of estrogen-like substrates. In addition to antibody molecules and antiidiotype antibody molecules, other sets of shape and shape-complement molecules, including DNA, RNA and other complex molecules can be used. These can, as one non-limiting example, be selected from high diversity combinatorial libraries of molecules.

In another embodiment of the present invention, a group of molecules are used which contain autocatalytic sets, e.g., autocatalytic sets of catalytic polymers. Reaction mixtures comprise such organic molecules which are simultaneously substrates and catalysts. Reactions are carried out in a chemostat under flow conditions. For example, molecules A, B and C are present wherein molecule B catalyzes its own formation out of substrate molecule A, and molecule C catalyzes its own formation out of substrate molecule A. This reaction is carried out in a chemostat where a receptor molecule, such as acetylcholine receptor is affixed to the walls of the chemostat and can bind any molecule that looks like acetylcholine. In this example, molecule B but not C looks sufficiently like acetylcholine to bind to the receptor for acetylcholine that is on the chemostat walls. Under flow conditions, the B molecule will tend to be selectively retained within the chemostat and the C molecule will not be retained. This provides selective conditions which leads to the selective amplification of the B autocatalytic set compared to the C autocatalytic set. For example, if B, even when bound to the receptor acts as a catalyst leading to its own formation, then its retention within the system is selectively favored, and B is amplified with respect to C. More generally, in a complex reaction mixture in which molecule B functions as a catalyst in its own formation out of the complex reaction mixture, then retention of B is selectively favored because it binds to the receptor for acetylcholine. Thus, in general, by taking a system under chemostat conditions in which one has a receptor for a molecule X, where finding analogs of X is of interest (X here is for example acetylcholine), then this is a general procedure to select among autocatalytic sets for those sets synthesizing X-like mimics. Hence, this selective method enhances the capacity to use random complex reaction mixtures to synthesize drug candidates able to mimic X.

In another aspect of the present invention, methods are provided for generation of new compounds without the use of enzymes. In one embodiment, the method comprises the steps of (a) reacting a group of different substrates, the group comprising acids, amines, alcohols, and unsaturated compounds, under suitable conditions with a dehydrating agent to yield a first reaction mixture; (b) reacting the first reaction mixture with a reducing agent under suitable conditions to yield a second reaction mixture; (c) reacting the second reaction mixture with an oxidizing agent under suitable conditions to yield a third reaction mixture; (d) performing a condensation reaction under suitable conditions upon the third reaction mixture to yield a fourth reaction mixture; (e) exposing the fourth reaction mixture to light of wavelength of about 220 nanometers to 600 nanometers, thereby producing one or more organic molecules different from the substrates and agents; (f) screening the exposed fourth reaction mixture for the presence of an organic molecule having a desired property; and (g) isolating from the exposed fourth reaction mixture the organic molecule having the desired property.

In another embodiment, the method comprises the steps of: (a) reacting a group of different substrates, the group comprising acids, amines, alcohols, and unsaturated compounds, under suitable conditions with a dehydrating agent to yield a first reaction mixture; (b) reacting the first reaction mixture with a reducing agent under suitable conditions to yield a second reaction mixture; (c) reacting the second reaction mixture with an oxidizing agent under suitable conditions to yield a third reaction mixture; (d) performing a condensation reaction under suitable conditions upon the third reaction mixture to yield a fourth reaction mixture; (e) exposing the fourth reaction mixture to light of wavelength of about 220 nanometers to 600 nanometers, thereby producing one or more organic molecules different from the substrates and agents; (f) screening the exposed fourth reaction mixture for the presence of an organic molecule having a desired property; and (g) determining the structure or functional properties characterizing the organic molecule having the desired property.

In this aspect of the present invention, a group of different substrates, such as those described above, are subjected to a series of reaction conditions from which one or more compounds having a desired property are produced without the use of enzymes. More specifically, a group of different substrates are reacted under suitable conditions with a dehydrating agent to yield a first reaction mixture. Suitable dehydrating agents include carbodiimides, carbonyldiimidazole, sulfonyl halides, phosgene equivalents and activated phosphoramides, as well as other agents in common use for solid phase peptide synthesis and nucleotide synthesis, etc. It will be evident to those of ordinary skill in the art that the most preferred solvent(s) are dependent upon the particular group of substrates selected. For example, if all the substrates are fairly polar in nature, a solvent such as methanol may be used. Concentrated solutions of individual substrates are made and then the group of substrates prepared by mixing aliquots of each concentrated solution. Mixtures of solvents which are miscible with one another (i.e., do not form two phases) are appropriate where all the substrates are not soluble in a single solvent. Examples of solvent mixtures are acetone and water, dimethyl formamide and water, or ethanol and water. Reaction conditions may be varied, but generally the reaction will be performed from about one hour to overnight at a temperature from about room temperature to the boiling point of the solvent.

The first reaction mixture, such as that described above, is reacted under suitable conditions with a reducing agent to yield a second reaction mixture. Suitable reducing agents include dissolving metals, hydride reagents, molecular hydrogen with suitable metal catalysts (e.g., platinum, palladium, nickel or rhodium), etc. Examples of reducing metals include sodium, lithium, potassium, various amalgams, calcium, iron, and tin. Examples of hydride reagents include sodium borohydride, lithium aluminum hydride, and borane. Reaction conditions may be varied, but generally the reaction will be performed from about one hour to overnight at a temperature from about room temperature or below (e.g., in an ice bath). It will be evident that certain reducing agents perform best in certain solvents. For example, where hydride reagents (such as sodium borohydride) are used, it will be evident that non-hydroxylic solvents (such as dimethylformamide) are preferred.

The second reaction mixture is reacted under suitable conditions with an oxidizing agent to yield a third reaction mixture. Suitable oxidizing agents include ozone, peroxides, chromate, permanganate, osmium tetroxide, chlorine, bromine, and air in the presence of suitable metal catalysts (such as ruthenium tetroxide). Reaction conditions may be varied, but generally the reaction will be performed from about 1-2 hours to overnight at a temperature from about room temperature or below (e.g., in an ice bath). It will be evident that certain oxidizing agents function best in certain solvents. For example, a mixture of water and alcohol may be used with hydrogen peroxide, but water only with permanganate, and hexane (or petroleum ether) with halogens such as chlorine or bromine.

A condensation reaction is performed under suitable conditions upon the third reaction mixture to yield a fourth reaction mixture. The third reaction mixture may be subjected to condensation by dehydrating agents or heat. Suitable dehydrating agents include molecular sieves, carbodiimides, azeotropic distillation (to remove water), etc. For example, toluene may be added and then azeotropic distillation performed to remove water. It will be evident that reaction conditions vary depending upon the type of dehydration agent used.

The fourth reaction mixture is exposed to light. The light generally is within a range of about 220 nanometers to 600 nanometers, which includes portions thereof or discrete wavelengths if desired. Reaction conditions may be varied, but generally the irradiation of a reaction mixture will be performed from about 15 minutes to 2 hours at a temperature from about room temperature or below (e.g., in an ice bath).

All the above-described reactions are generally performed at ambient pressure. Certain exceptions, such as reduction using molecular hydrogen, will be evident. It will be readily appreciated by those of ordinary skill in the art that a group of substrates may be subjected to the various reaction steps in orders which differ from the order provided above. Further, it will be evident that after subjecting a group of substrates to any one or a subset of the various reaction steps above in any order one or more of the steps may be repeated if desired. Further, it will be clear that other reagents, used singly, or in mixtures, or used sequentially, in addition to the above examples, or with the above examples where practical, can be utilized. The repetition of steps need not be in the order initially performed and additional substrates may be introduced at any step if desired. In addition, one or more of the substrates used initially, or introduced at a subsequent reaction step, may be generated by any of the methods provided herein, i.e., by random chemistry with or without enzymes.

As described above, in an embodiment of this aspect of the present invention, the group of substrates is provided by derivatization of an initial molecule or a class of molecules. Such a group of substrates is subjected to the above-described reactions without enzymes to generate a high product diversity which is centered around the initial molecule or a class of molecules.

A variety of means are available which allow detection of low concentrations of one or more species of a desired molecule in a mixture of molecules generated by the methods provided herein. For example, a variety of cell systems are well known to those of ordinary skill in the art which allow detection of low concentration ligands, e.g., ligands binding a hormone receptor. In this regard, for example, a system has been developed which clones human G peptide hormone receptors into frog melanocytes (Lerner, Proc. Natl. Acad. Sci. USA). The hormone receptors, typically located in the cell membrane, respond to binding of the corresponding hormone, but trigger a cell response releasing or reabsorbing melanophores. In a forty minute reversible cycle, cells darken dramatically, then can be induced to lighten in color again. Response of the cell depends upon the affinity of the hormone for the receptor. Typical responses occur in the nanomolar to 100 picomolar hormone concentration range. For some hormone receptor-hormone pairs, where affinity is higher, response occurs in the picomolar hormone concentration range. This cell system is an example of an assay system which allows detection, in a mixture of molecules, of one or more species of ligands able to bind to the receptor. The set of molecule ligands able to bind the receptor are then the ligands of interest, for they are candidates to act as drugs by antagonizing, agonizing, substituting for, or modifying the effects of the natural hormone.

A second example of a cell assay is that available commercially from Molecular Devices (Palo Alto, Calif.). It consists of an array of chemfets which respond to very small changes in local pH. In turn, these small pH changes reflect the altered metabolic activity of a population of cells upon receipt of some molecular signal, such as a hormone binding its receptor. For example, cell assays in which a hormone binds a receptor are known to those of ordinary skill in the art and allow nanomolar or subnanomolar concentrations of the hormone ligand to be detected. A preferred means of using the present invention consists in exposing such cells to a high diversity library of molecules generated by the methods provided herein, to detect the presence of one or more species of molecules able to trigger the cell response. That set of small molecules, each of which is highly likely to bind the hormone receptor, are the molecules of interest which may serve as drugs. Another example is to use blast B cells, which on their surface express antibodies directed to a molecule of interest, to detect in a high diversity library the presence of molecules which sufficiently mimic the molecule of interest to be able to bind to its antibody on a B cell. Thus, an animal is immunized with a molecule of interest and the early B cells isolated. A high diversity library of molecules generated by the methods provided herein is screened using the population of B cells. For example, binding may stimulate cell cycling or division by the last B cell bound. Cell cycling or division may be detected by means known in the art.

Alternatively, a variety of assays to detect the presence of a ligand of interest exist which are based on direct binding assays. Thus, for example, a receptor for a hormone can be used directly to detect binding of a radioactivity labeled ligand. Other means, known in the art, to accomplish this include the following:
(i) The estrogen receptor is used as a non-limiting example. The cloned receptor can be affixed to a flat surface, for example, a filter. Very high specific activity estrogen is prepared, and bound to the receptor population. This set of bound receptors is then used in a competitive assay. The bound receptors are exposed to a library of compounds generated by the methods of the present invention. If the library contains ligands which also bind the estrogen receptor, those ligands will compete with the radioactively labeled estrogen itself for the receptors. Hence the radioactively labeled estrogen will be competitively displaced from the receptor, and can readily be detected by means known in the art. Thus, this assay allows detection of one or more species of ligands in the mixture which compete with estrogen for the estrogen receptor. This set of ligands is the set of interest, as they are candidates to be drugs mimicking or antagonizing estrogen.

(ii) The estrogen receptor is again used as a non-limiting example. By means known in the art, one raises antibody molecules which are able to bind the receptor when the receptor is not bound by estrogen, but not bind the receptor when occupied by estrogen. Alternatively, one generates antibody molecules which bind the estrogen receptor only when the receptor itself does bind estrogen. These antibody molecules can then be decorated with reporter groups by a variety of means known in the art, and used to detect the presence of one or more ligand species in a library of high diversity, which bind to the estrogen receptor. In the case of antibodies which only bind the receptor if the receptor is itself unbound by estrogens, one tests for loss of antibody binding in the presence of the library of compounds and in the simultaneous absence of estrogen. In the case of antibodies which bind the receptor only if the receptor is bound by estrogen, one tests for an increase in binding of the antibody in the presence of the receptor and high diversity library.

(iii) In order to detect ligands in a high diversity library which are candidates to mimic or antagonize the action of a given hormone or other molecule of interest, it is advantageous to generate one or more monoclonal antibodies which bind the hormone or other molecule of interest. This set of monoclonal antibodies can then be used, rather than a receptor, for the target molecule that is to be mimicked, in binding assays such as those noted above to detect the presence of one or more ligand species in the reaction mixture which are candidates to mimic or antagonize the action of the target molecule.

An advantage of this procedure is that a receptor for the target molecule need not be available. Use of a set of monoclonal antibodies is advantageous because, a priori, it is not certain which molecular feature, or epitope, of the target molecule mediates its biological action. Use of a set of monoclonal antibodies, each responding to a different epitope on the target molecule, enhances the probability that the ligands detected in the high diversity library will include those which mimic the biologically important epitope of the target. In some cases it may be possible to selectively use only those monoclonal antibody molecules which bind to the known important epitope of the target molecule.

(iv) Means are established in the art to detect protein-protein binding based on plasmon resonance and detection of a shift in refractive index. In a detection system developed by Pharmacia (Piscataway, N.J.), a monoclonal antibody, or a hormone receptor, is layered onto a gold chip. Binding of hormone, or other ligands to a receptor, is detected in very low concentrations (e.g., in the nanogram range or less). Thus, any receptor, or antibody, or other “shape complement” of a target molecule of interest can be placed on the gold chip, the latter can be exposed to a high diversity library, and the presence of liganding species can be detected.

Another example of direct measurement of ligand-binding, which the applicant believe was developed by Evotech, can measure ligand binding in the femtomolar range. Rudolph Rigles of the Karolinska Institute in Stockholm has described a laser assay system in which a laser is focused on an approximately 1 cubic micron volume of fluid, and can detect the presence of fluorescently labeled compounds at femtomolar concentrations, 10⁻¹⁵ M, in tens of seconds. By fluorescent labeling of small “shape-complement” molecules of a desired target molecule, the binding of a target-mimic molecule to the shape-complement can be detected through alteration of the diffusion of the ligand-bound versus free shape-complement molecule. Thus, if estrogen is the target molecule, and a small RNA aptomer is the shape-complement which binds estrogen, then fluorescent labeled versions of that RNA aptomer can be used in Rigler's system. An estrogen-mimic which binds the fluorescently labeled RNA will slow its diffusion as detected in the laser system. Thus estrogen-mimics at very low, 10⁻¹⁵ M or femtomolar, concentrations can be detected.

A further means to detect ligands of interest at very low concentrations consists in seeking ligands which block a DNA polymerase. By blocking the DNA polymerase chain reaction (PCR) enzyme, amplification of the DNA can be blocked. Since PCR amplification can yield billions or more copies of the initial DNA sequence, blocking PCR amplification yields a readily detectable signal of a ligand which blocks the polymerase. Clearly, this method generalizes to other means to amplify DNA, RNA, or DNA- or RNA-like molecules such as ligation amplification, and extends to general means to block polymerases directly or indirectly with ligands of interest.

Given that the diversity of the library of molecules which must be tested for molecules of interest is related inversely to concentrations and given that the requirement that the founding substrates must be jointly soluble in the reaction mixture, then driving the detection level to very low concentrations permits the invention to be utilized to explore libraries of extremely high diversities. Diversities of 10¹⁵ can be generated, and the presence of ligands of concentrations of 10⁻¹⁵ to 10⁻¹⁶ M can be both detected and generated from initial millimolar mixtures of 1,000 to 100 substrates. Additionally, with a sufficiently high diversity of enzymes or reaction conditions, a high diversity library may be generated with a founder set of organic compounds with a diversity as small as 10.

As described above, compounds of interest in the high diversity library may act as catalysts for a desired reaction, or as cofactors with other molecules to form an active catalyst. Other molecules may act as inhibitors of enzymes. In order to exclude the possibility that the enzymes or catalysts are found among the candidate set of enzymes which may have been used to generate the library, the latter set of enzymes can be quantitatively removed from the high diversity library by affinity columns bearing molecules directed to a constant part of each of the set of enzymes, or other means known in the art. The resulting high diversity library itself is then assayed for candidates of interest.

Detection of molecules able to inhibit an enzyme may proceed by detecting ligands able to bind the enzyme, as described above. Identifying molecules which are candidates to catalyze a reaction alone or as a cofactor, may proceed by testing high diversity libraries alone, or in the presence of a helper molecule, say a protein, for which a desired molecule will be a cofactor. The system is tested for the presence of ligands able to bind a stable analogue of the transition state of the reaction. Such binding molecules are the candidate catalysts or cofactors sought, for they are candidates to catalyze the reaction itself.

Alternatively, a variety of means are known in the art which allow detection of the products of a catalyzed reaction itself. For example, chromogenic or fluorogenic substrates for a variety of reactions of interest are available. Catalysis of the reaction increases the rate of formation of the colored or fluorescent product. Alternatively, assay systems are available or readily prepared which detect the presence of a product molecule because that product molecule binds a receptor, an antibody molecule, or other shape complement. Thus, detection of higher rates of formation of that product molecule demonstrates that the reaction itself was catalyzed.

Following the generation of high diversity libraries of compounds and the screening for the presence of compounds having properties of interest, such compounds of interest are characterized with or without isolation. A variety of means, including those known in the art, are available to characterize or isolate such compounds of interest.

Characterization and/or isolation, depend upon the information desired, and can be carried out at different mole abundances of the target molecule of interest. Thus, using modern mass spectrograph analysis, about 10⁻¹⁵ to 10⁻¹⁸ moles can be assayed for mass and charge, then fragmented in a variety of ways known in the art and the fragments assayed for mass and charge. Using this data, it is possible to derive the structure of the molecule of interest. For example, ligands of interest may be isolated by binding to a given hormone receptor, or monoclonal antibody, then the liganding molecules released by means known in the art, and finally characterized analytically. One means comprises attaching a target receptor or antibody to a solid support. A reaction mixture or subset thereof is contacted with the solid support. Those molecules that are bound will be retained, while the non-bound molecules are readily separated from the solid support. The molecules of unknown structure which have been retained, are then eluted. The freed molecules are characterized analytically, e.g., by mass spectroscopy, NMR, IR, UV, and may be synthesized in batch quantities. Examples of analytic techniques involving mass spectrometry include gas chromatography-mass spectrometry (GC-MS), HPLC-mass spectrometry (LC-MS), and field desorption mass spectrometry (FD-MS).

In other cases, the concentrations of molecules of interest in the high diversity library will allow detection of their presence, but may be too low for further isolation or characterization. A preferred procedure called “sib selection” allows ready winnowing of the set of candidate enzymes, the set of founder substrates, and the set of reaction conditions and chemical reagents, to smaller sets. This winnowing simultaneously reduces the side products generated in the high diversity library, increases the concentration of the target molecule of interest, and identifies the subset of candidate enzymes which catalyze the pathway leading to synthesis of the target molecule, and identifies the set of founder substrates required for synthesis of the desired target. Thus, this sib selection procedure is a means to generate a previously unknown molecule of interest, as well as identify both that molecule and the substrates and enzymes needed to form that molecule.

A library, where the target of interest is a molecule which binds the estrogen receptor, is used as a non-limiting example. For example, a high diversity library derived from D and L amino acids, including nonnatural amino acids, and small peptides which may be composed thereof is provided by the methods described herein. Such a library will contain linear, branched, cyclic and other singly or multiply constrained forms due to formation of disulfide (S—S) intramolecular bonds.

An aspect of the present invention where substrates and candidate enzymes are used is discussed first. Further below, another aspect of the present invention where candidate enzymes are not used, but one or more reagents or reaction conditions are used, is discussed.

The presence of one or more ligands for the estrogen receptor is detected in the high diversity library of this example by any of the means described above, or any other means. The set of candidate enzymes and set of founder substrates suffice to lead to reactions which generate the desired ligands. As a non-limiting example, a set of four reaction steps, using seven of the initial substrates at different reaction steps, may lead to the desired target molecule. By winnowing down the set of initial substrates to the seven needed, and the set of four enzymes needed, the target molecule may be synthesized in high concentrations. High concentrations may be achieved because, given the solubility limits, higher concentrations of the seven critical substrates may be attained than when 1,000 initial substrates were used, and because only the four critical enzymes would be present.

Sib selection achieves this winnowing. One may start with the candidate set of enzymes, but could equally easily start winnowing the set of substrates. The set of candidate enzymes can be derived, for example, from a cloned polynucleotide library. Thirty-two aliquots are created, each of which contain a random half of the initial diversity of the candidate enzyme library. Thus, if the initial enzyme library diversity was 1,000,000, thirty-two aliquots are created, each containing a diversity of 500,000 candidate enzymes. The chance that any aliquot has the four critical enzymes is therefore 1/16. Hence, on average, 2 of the 32 aliquots have the four critical enzymes. The full set of initial substrates are added to each aliquot, the reactions run, then each aliquot tested for the presence of the desired target molecule which binds the estrogen receptor. One or two of the aliquots are positive. Each of these aliquots has decreased the diversity of candidate enzymes by a factor of two, from 1,000,000 to 500,000. One of the aliquots which is positive is chosen. The other can be stored for later analysis. Again 32 aliquots are created, each again having a random half of the remaining candidate enzyme diversity. Hence each of the 32 aliquots now has a diversity of 250,000 candidate enzymes. Each is again tested for formation of the target molecule which binds the estrogen receptor. Therefore, in a logarithmic number of iterations, the set of candidate enzymes may be winnowed down to the four needed to catalyze the synthesis of the target molecule. In the present case about 18 iterations are required.

This winnowing procedure, therefore, allows the isolation of a set of enzymes needed to synthesize a target molecule of interest. Thereafter, mutation, recombination and selection can be used on this set of enzymes to increase their efficiency and specificity in producing the target molecule. Thus, this procedure yields an efficient set of enzymes for later synthesis of the target molecule from its progenitor substrates. In a further use of the present invention, mutant forms of these enzymes can be utilized to catalyze a related family of reaction steps leading to variant forms of the target molecules. Those variants may be more useful than the initial molecules.

In this example, the set of substrates may also be winnowed to the seven needed. This winnowing can occur either before or after the set of enzymes is winnowed. The process is the same. Thirty-two aliquots are created, each containing a random 80% of the 1,000 initial substrates. The chance that any aliquot contains the seven critical substrates is 0.8⁷. Thus, on average one or more of the aliquots contains the requisite set of 7 substrates. Each aliquot is tested for the presence of the target molecule of interest that binds the estrogen receptor. A positive aliquot is chosen. Thirty-two aliquots are again generated, each containing a random 80% of the remaining, now reduced substrate diversity. The aliquots are again tested for those which contain the target molecule of interest. In a logarithmic number of steps it is possible again to winnow to the seven critical initial substrates. The number of steps is modest.

It is clear that the fraction of the candidate enzymes or initial substrates used in each aliquot at the first winnowing step, and each step thereafter, can be chosen such that the expected number of aliquots which form the desired molecule is one or greater than one at each step of the winnowing process.

In modes of generating a high diversity library where no candidate enzymes are used, but one or more reaction conditions and reagents are used, the set of initial substrates may be winnowed using the sib selection procedure described above. This increases the concentration of the target molecule because the diversity of molecules present and resulting side reactions is sharply reduced. In addition, in advantageous cases it may be possible to winnow out those reagents or physical conditions not needed to synthesize the target molecule.

One aim of the sib selection procedure is to obtain a sufficient abundance of the target molecule for its characterization and synthesis by independent means known in the art. Typically, microgram or milligram quantities are sufficient for such analysis by standard techniques. As noted, it may often be possible to deduce structure and composition from far smaller quantities by mass spectrographic analysis or other means known in the art.

It will be appreciated that it is not necessary to actually isolate a compound to homogeneity from a reaction mixture, where sufficient information about the compound or its functional properties can be accumulated in its less than purified state. For example, sufficient structural information may be obtainable using analytical techniques appropriate for mixtures of compounds. Alternatively, a compound in a reaction mixture may be characterized functionally (e.g., it is defined by the set of molecules with which it is capable of interacting). For example, a compound in a reaction mixture may interact with a particular amino acid or small sequence of a polypeptide, resulting in enhanced or diminished function of the polypeptide. For example, the compound might be a suicide substrate which covalently links to a polymer near the catalytic site. Such a bound suicide substrate may be used to identify catalysts with a desired activity, or to characterize features of the active site of such a polymer. The site of interaction on the polypeptide may be detected by analytic techniques which are capable of detecting perturbations to individual amino acids or regions of the polypeptides. This information regarding the locus for alteration of the polypeptide's function (i.e., information about the target) may be equally or more important than the structure of the compound in the reaction mixture which interacted with the polypeptide. It will be evident that, based on this type of information, one may modify a particular amino acid or region of a polypeptide in a variety of ways.

The following examples are offered by way of illustration and not by way of limitation.

### EXAMPLES

### Example 1

### Preparation of Ubiquitin Fusion Libraries With Diversity of 1×10⁷

The single-stranded DNA needed for 38, 71, and 104 amino acid polypeptide libraries is synthesized. The total diversity is on the order 10¹⁵. PCR amplification is carried out by routine methodology. Ligation and transformation efficiency, without attempts to optimize, ligates on the order of 10⁷ random sequences into plasmid, and after transformation yields about 30,000 clones. An efficiency yielding of about 10,000,000 to 100,000,000 transformants per ug of plasmid DNA is attainable (Sambrook et al., Molecular Cloning: A Laboratory Manual, 2d ed., Cold Spring Harbor Laboratory Press, 1989). Using 50 ng per transformation, 500,000 to 5,000,000 clones per transformation is achieved. Transformation may be optimized by (i) purifying the insert DNA, (ii) optimizing ligation conditions, or (iii) optimizing transformation technique and conditions. Even at unoptimized efficiency, a polypeptide diversity of 1,000,000 with thirty transformations is attained.

On average, each sequence among the 10′ ligated is unique. The diversity obtained is tested by counting total transformants created, sampling random ampicillin resistant clones, carrying out plasmid preparations, restriction mapping and screening for inserts. This allows calculation of the total number of transformed clones obtained, but, since any sequence might be present in multiple copies, the total alone does not yet specify the total diversity.

Clone redundancy in the library is tested using plasmid preparations of a pool of 5,000 plasmids. Redundancy among these distinct plasmids is tested via hybridization with the unique random DNA region from each of several specific plasmids among the 5,000. To carry this out, 5,000 transformed colonies are grown on a single plate, lifted onto nylon filters (GeneScreen Plus, DuPont), the cells lysed, the DNA is UV-crosslinked to the filter, washed, the DNA denatured with NaOH, and then neutralized. Thereafter hybridization is carried out under stringent conditions with radiolabeled unique DNA probes purified from each of several plasmids among the 5,000. Probe DNA is cut from the adjacent ubiquitin sequences and gel purified prior to labeling. Probe is labeled by random primer labeling (Prime-It, Stratagene Cloning Systems). Autoradiography of the resulting filters reveals if any insert DNA sequence occurs in an expected one, or many among the 5,000 colony diversity on the plate. Given the distribution of numbers of colonies bound for each of 10 to 20 probe insert DNA sequences, the expected diversity of the library may be calculated based on maximum likelihood methods.

### Example 2

### Generating A Diversity of Product Molecules

The combinatorics of the libraries described in Example 1 are tested for the onset of catalyzed reactions as libraries of polymers act on one another. The number of possible interactions is enormous.

For example, for ligation reactions involving two DNA substrates and one polypeptide catalyst, the combinatorics admit of 10²¹ possibilities of interactions in the DNA and peptide libraries of a 10,000,000 diversity. Even where the probability that an arbitrary polypeptide catalyzes a given ligation reaction is 10⁻⁹ (an estimate based on the ease of finding catalytic antibodies), a very large number of distinct reactions are catalyzed. Although the combinatorics favor the onset of catalyzed reactions, as the diversity of reactants increases, the concentration of any type of sequence decreases proportionally. For bimolecular reactions, the forward rate decreases as the square, for trimolecular reactions the rate decreases as the cube of the falling concentrations.

Using an estimate of the probability of catalysis of 10⁻⁹ and seeking two substrate reactions such as ligation, transesterification, or transamination to score, the desired product concentrations and catalyzed reactions may be achieved with diversities of 10⁴ in both the DNA and polypeptide libraries. For unimolecular reactions such as cleavage, or phosphorylation, diversities on the order of 10⁵ to 10⁶ in both the substrate and catalyst library are needed.

A first set of experiments utilizes single stranded DNA sequences as substrates. Subsequent experiments use polypeptides as substrates. This choice is made for three reasons. First, production of novel DNA sequences, whose length differs from the initial set of substrates, all of identical length, is easy to detect on sequencing gels. Second, single stranded DNA, like RNA, is able to fold into complex structures (Lu et al., J. Mol. Biol. 223:781-789, 1991), hence afford a wider variety of sites for binding and catalysis than double stranded DNA sequences of the same total diversity. Third, single stranded DNA is easier to obtain from the libraries than the corresponding RNA, and somewhat more stable against degradation. Nevertheless, RNA of high diversity specified by the libraries may be purified. Alternatively, DNA sequences may be modified to include RNA polymerase premier sites such as T7 to allow in vitro RNA transcription (Ellington and Szostak, Nature 355:850-852, 1992), and obtain high diversity RNA libraries for use as substrates. Thus, protocols are stated in terms of single stranded DNA substrates, but single stranded RNA libraries may also be used.

The plastein reaction (Wang et al., Biochem. Biophys. Res. Commun. 57:865, 1974; Silver and James, Biochemistry 20:3177, 1981) is a general model for the experiments. In this reaction, protein substrates are incubated with trypsin, which cleaves the substrates to smaller peptides. Since any enzyme catalyzes forward and reverse reactions, trypsin is capable of catalyzing ligation of larger polypeptides from the smaller peptide fragments. It has been found that dehydrating the reaction mixture, to shift the equilibrium in favor of synthesis, suffices for trypsin to catalyze ligation and transamination reactions leading to formation of high molecular weight polypeptides in the absence of ATP hydrolysis (Levin et al., Biochem. J. 63:308, 1956; Neumann et al., Biochemistry 73:33, 1959). If the high molecular weight material is removed and the reactants again concentrated, further high molecular weight polypeptides are formed. Absence of a requirement for ATP hydrolysis is not too surprising, since transamination reactions can proceed without net formation of new peptide bonds.

In the first set of experiments, single stranded DNA sequences of constant length from the libraries, end labeled after the reaction in one set of experiments, and uniformly labeled prior to the reaction in another set of experiments, are incubated with ³²P nucleotides. The substrates are then incubated with affinity purified polypeptides from the libraries of Example 1 with length 38, 71, and 104 of tuned diversities' in the ranges noted. Divalent cations, such as Mg++, Pb++, Mn++, as well as ATP as a potential energy source may be included. In addition, concentrations of DNA substrates and polypeptides are tuned over a range sufficiently broad to include conditions under which biological polynucleotides are cleaved or are ligated in vitro. In ligation reactions, typical DNA “ends” concentrations are nanomolar. In a variety of reactions, typical enzyme concentrations are micromolar or higher. DNA substrate ranges in the nanomolar concentrations are easily created under the present experimental conditions. For polypeptides from the 71 amino acid library, a diversity of 10,000 polypeptides at 1.0 mg/ml yields about a 10.0 nanomolar concentration for each fusion protein. Therefore, reactions catalyzed efficiently by such novel enzymes, produce product 100 times slower than were their concentrations higher. In typical DNA cleavage or ligation reactions, substantial product can be detected after times on the order of minutes. Thus, in general, detectable products are seen on the order of hundreds of minutes to thousand of minutes.

The polypeptides in the library catalyze, for example, cleavage, ligation or transesterification reactions among the single stranded DNA target molecules. Of these, cleavage is energetically favored in an aqueous medium, while transesterification reactions, like transamination reactions among polypeptides in the plastein reaction, are nearly constant energetically in aqueous media. In addition, a variety of crosslinking reactions between two single stranded substrates may occur. Transesterification reactions between two substrate sequences of length L can yield two product molecules, one of which is larger than either of the two substrate sequences. The beginning library of DNA molecules are all of the identical length. Thus, on a large 38 cm polyacrylamide gel (BRL Sequencing Apparatus) run under denaturing conditions, the entire library runs as a single band. However, where the polypeptides catalyze cleavage, ligation, transesterification or crosslinking reactions with DNA molecule substrates, new shorter or longer DNA sequences appear on the gel. Using standard DNA sequencing on long gels, bands which differ by a single nucleotide can be discriminated over about a 400 base range. The gel is run to adjust the position of the random library full length single stranded DNA sequences at a desired position on the gel. Using aliquots of the same reaction mixture sampled at the same moment, and running gels for different durations, a large range of molecular weights are scanned for novel bands. As noted, all products of reactions in one set of experiments are end labeled, since uniform labeling of substrate sequences prior to reaction with ³²P may induce radiation breaks in single stranded substrates. The end labeled material should be stable, but less label is present on the gel, rendering detection more difficult, and only one fragment of a cleavage reaction is visible. Uniform labeling achieves higher specific activity and legitimately marks reactions yielding product molecules which are larger than our single stranded substrates.

In order to assure that the new molecular size classes represent de novo catalysis due to the polypeptide library, control reactions are carried out using a control library encoding ubiquitin alone. If affinity purified ubiquitin alone, derived from the control library, catalyzes reactions among the DNA substrates, then this can be controlled for in two ways. First, novel random peptides are cleared free from ubiquitin as noted above, the novel peptide fragments repurified by size under non-denaturing conditions, and retested for catalysis using these random peptides freed of ubiquitin. Second, the particular reaction substrates acted on by ubiquitin or cell background material can be identified by a logarithmic dilution technique, as described below, and eliminated from the DNA substrate library.

A number of features of this system may be assessed. First, the probability that a protein catalyzes a detectable reaction on DNA substrates may be estimated. At low diversities of the libraries, the appearance of a few distinct bands of lower or higher molecular weight than the initial DNA substrate library may be seen. Where these are the only reactions catalyzed, then as the incubation period increases, no further bands appear. Each cleavage reaction involving a single DNA substrate may give rise to two product sequences. Transesterification reactions between two substrates again give rise to two product sequences per reaction. Crosslinking and ligation reactions yield one new product sequence. Single crosslinking and end ligation reactions yield one new product sequence. Single crosslinking and end ligation reactions among a uniform set of single stranded DNA sequences length L should all have a total length of 2L nucleotides. Therefore, for new bands corresponding to lengths less than 2L, the number of reactions is estimated as half the number of such new bands. Using this data, one may estimate the probability that an arbitrary polypeptide catalyzes a detectable reaction. (Some crosslinked DNA sequences with 2L nucleotides may have aberrant migration characteristics, perhaps leading to erroneously count them as products of transesterification reactions. This could cause a two-fold error in the estimated probability.) Second, this estimated probability may be confirmed by increasing the substrate and polypeptide diversity. Third, by tuning polymer length at constant diversity, the effective number of substrate sites and of catalytic sites may be measured as a function of polymer length.

In an additional set of experiments to test whether the set of polypeptides catalyze reactions, unlabeled single or doubled stranded DNA sequences of constant length derived from the libraries is incubated with ³²P labeled nucleotides or short oligonucleotides, acrylamide gels run, and the labeled material is tested for incorporation into large molecular weight DNA material.

A new general “logarithmic dilution” procedures is carried out to isolate both the specific polypeptide(s) catalyzing any specific reaction, and the specific substrates involved. The procedure introduced here also serves to isolate both the specific set of substrates and the specific set of novel enzymes leading to the synthesis of a target molecule of interest.

To carry out this procedure, divide the total diversity of the initial cloned polypeptide library into four different aliquots, each containing a random half of the total diversity of the polypeptide library. Aliquots may be created which reduce total diversity by random halves by knowing the diversity of the library, and the number of copies of each sequence by methods known to those skilled in the art

For reactions with two substrates and one enzyme, the probability that any random half of the diversity of the polypeptide library has the requisite enzymatic polypeptide is 0.5. Thus, two of the set of four random half-library aliquots contains the required polypeptide. If no random halved aliquot had the required polypeptide, a larger number of halved aliquots is tested. Each new diminished library is incubated with the full set of single stranded DNA substrates, and the products analyzed on a long sequencing-type gel. On average, for two such gels, the desired product of the reaction continues to be present. Thus, the corresponding half polypeptide library contains the polypeptide which catalyzes the reaction. That now diminished library is again divided into four random halves in four aliquots. Each is incubated with the full set of DNA substrates, the gel run and the product identified if formed in at least one of the four aliquots. By a logarithmic number of halvings of the initial polypeptide library, the single polypeptide catalyzing a specific reaction is isolated. Simultaneously, the fusion gene encoding this polypeptide is isolated. Thus, if the polypeptide diversity is on the order of 10,000, then about 13 halvings suffice.

In the same way, the specific substrates for the reaction in question are obtained. For two substrate reactions, eight random halves of the DNA substrate library are progressively formed. The probability that any aliquot contains the two substrates is 0.25, hence on average two of the eight have the two substrates. These aliquots with the now known catalytic polypeptide are incubated, gels run, which aliquot exhibits the desired reaction product confirmed, thereby concluding that the corresponding half of the substrate diversity contains the desired two substrates. Over a logarithmic number of successive rounds, the two substrates are thus isolated.

As noted, a main virtue of this approach is that it is possible to carry it out for any set of molecule substrates, and any set of polypeptide, RNA, or other potential catalysts. In short, where a diversity of new products are formed under these experimental conditions, and where one such product is of interest and can be reliably found in the product mixture after reaction, then a modest number of halving steps isolates both the substrates for and enzymes for the reaction leading to the product. This approach generalizes to cases in which several enzymes carry out a succession of reactions from an initial set of substrates. It is merely necessary to alter the random fraction of the diversity in each aliquot, and number of aliquots at each step, to assure that at least one such aliquot contains the requisite set of substrates or enzymes. At any diversity, a logarithmic number of steps is required to isolate both the set of substrates and the set of enzymes leading to synthesis of a desired novel target compound.

The polypeptide libraries of tuned diversity may be permitted to act on themselves as substrates. Many of the same considerations apply to polypeptide and DNA sequences as substrates for reactions. Cleavage is energetically favored in aqueous medium, while transamination reactions are energetically neutral. Thus, as noted, in the plastein reaction, increasing the concentration of the peptide fragments by dehydration shifts the transamination reactions in favor of synthesis of large molecular weight polypeptides, and the reactions proceed without ATP hydrolysis (Neumann et at., Biochemistry 73:33, 1959). Thus, after incubation of a set of labeled polypeptides of a constant length and mean molecular weight, formation of novel lower and higher molecular weight sequences may be seen. A variety of endoproteases, exoproteases and other enzymes may be used to drive the efficient synthesis of larger polypeptides from smaller peptide substrates. Enzymes used include subtilisin, papain, thermolysin, chemotrypsin, and carboxypeptidase Y, in enzyme concentrations ranging from micromolar to millimolar, and substrate concentrations ranging from millimolar to molar (Wong and Wang, Experientia 47:1123 1129, 1991).

Based upon a solubility of 1.0 mg/ml for the polypeptide fusion library, then at a diversity of 100, each 71 amino acid fusion peptide is present at approximately 0.6 micromolar concentration. With a diversity of 1,000,000, each is present at 0.06 nanomolar concentration. In a volume of 10 ml, a diversity of 1,000,000 corresponds to 10 nanograms of each. These concentrations are detectable. For example, gold stained blots on Immobilon P filters can detect spots with 3.5 nanograms, and polyacrylamide gel staining can detect bands or spots of 2.0 nanograms (Pluskal et al, Bio/Techniques 4(3):272-282, 1986; Ausubel et al., eds., Current Protocols in Molecular Biology, Greene Publishing and Wiley-Interscience, New York, 1987). Radiolabeling increases detectability by more than an order of magnitude (Garrells, Methods Enzymol. 254:7961-7977, 1979). In order to maximize substrate, hence product concentrations, the diversity and concentration of the polypeptide library may be tuned to find that minimum diversity and maximum concentration at which preferred new prominent bands appear. In addition to running one-dimensional SDS polyacrylamide gels, reaction mixtures are analyzed on two-dimensional gels, running first an isoelectric dimension, followed by SDS page analysis (O'Farrel, J. Biol. Chem. 250:4007-4021, 1975; Garrells, Methods Enzymol. 254:7961-7977, 1979; Summers and Kauffman, Developmental Biology 113:49-63, 1986). Automated facilities for digitized gel data analysis are available. Two-dimensional gels may be used to confirm that unique bands on one-dimensional gels correspond to unique spots in two dimension, hence a single product polypeptide. This allows one to count the number of reaction products.

For subcritical reaction systems of minimal diversity, only a few novel products are formed, and no further catalyzed reactions occur due to these new polymers. Thus, as incubation increases, no new bands or spots are generated. From the number of novel polypeptides produced, the probability that an arbitrary polypeptide catalyzes a reaction may be quantified. As above, cleavage and transamination reactions among polypeptide substrates length L typically yield two products of length less than 2L Ligation and crosslinking reactions yield one product with a total of 2L amino acids. Using two-dimensional gels, the number of distinct products of molecular weights corresponding to a total of 2L amino acids are discriminated, since one knows an expected mean molecular weight and a calculable variance. Thus, for a modest number of novel bands and spots, the total number of reactions catalyzed may be estimated. From this, the probability that a polypeptide catalyzes a reaction can be calculated. As the lengths of the polypeptides are altered, one may obtain measures of the scaling relation for numbers of types of reactions catalyzed as a function of polymer length of substrates and enzymes.

As noted above, phase transitions afford the ability to catalyze an explosion of molecule diversity from a diverse founder set of organic molecules acted upon by a sufficient diversity of potential catalytic polymers. Where target small molecules of interest are detected among the products of the catalyzed reactions, the logarithmic partitioning procedures above should allow the recovery of the specific substrates and novel enzymes leading to the molecule of interest.

In supracritical reaction systems, by definition, new products become substrates for yet further reactions engendering still further new products which again are candidate substrates. Three signatures are monitored to establish supracritical behavior. First, over time, the diversity of substrate and product species increases. This is the major criterion. Second, over time, the maximum molecular weight product increases. Third, the mean and variance in the molecular weight distribution among the products increases in a calculable way.

The second and third signatures require elaboration. In a supracritical reaction system where the initial substrate single stranded DNA, polymers are all of length L, the maximum length polymer which can be formed by a single ligation reaction is of length 2L. The maximum length which can be formed by use of two such newly formed polymers in a new ligation reaction where they are the substrates is 4L, then 8L and so forth. Thus, visualization of an increasing maximum molecular weight among the product molecules is evidence favoring supracritical behavior of the reaction system. More generally, in model reaction systems whose founder substrate sets are only a few monomers in length, the mean and variance in molecular weights among the product polymers increase over time and gives rise to a characteristic unimodal distribution. The diversity of polymers of a given length present in the system can be plotted on the ordinate and the lengths of those polymers on the abscissa. As reactions proceed creating a diversity of small and large products, the resulting curve may rise steeply to a peak as length increases, then fall off with an exponential tail.

In the first set of experiments, the diversity of new bands which appear on sequencing gels are analyzed as a function of time and as a function of the diversity of the polypeptide library catalyzing the reactions. In minimally diverse DNA substrate systems a modest number of new products may appear early, then not increase over time. In systems with a substantially higher diversity of single stranded DNA substrate sequences, detection of a sustained increase in total diversity over time (as limited by the product concentrations required for detection) and detection of a sustained increase in the highest molecular weight classes seen, are strong evidence for supracritical behavior of the reaction system.

In a second set of experiments, forward reaction velocities are driven, and the reaction system maintained in non-equilibrium conditions, by sustaining the concentration of the founder set of single stranded DNA sequences through periodic or continuous addition of labeled single stranded DNA sequences forming that set. Sustained non-equilibrium conditions through “driving” by addition of founder substrate molecules may be important to achieve high concentrations of high molecular weight polymers. The catalyzed reactions funnel monomers to specific large polymers.

Addition of founder substrate DNA polymers is carried out in two ways. In the first way, substrates are added to an otherwise closed stirred reactor. In the second way, substrates are added to a flow chemostat. The two environments are quite different. In a closed stirred reactor, product molecules are not removed from the system except by back reactions or further reactions in which they are substrates. In a flow chemostat, product molecules are removed. As shown in detail by Eigen and Schuster (The Hypercycle: A Principle of Natural Self-Organization, Springer-Verlag, New York, 1979), the chemostat system driven by continuous addition of substrate molecules is an environment which carries out selection on the reaction products: The total mass of substrate nucleotides ultimately becomes constant. The fraction of these which are organized into product molecules of different sizes may change. Those product molecules which are produced faster than they are diluted by the outflow actually accumulate in concentration, the remainder are gradually eliminated. Thus, the closed reaction system allows one to test for the total increase in product diversity over time. The flow chemostat environment allows one to test, as a function of flow and driving rates, whether the reaction system settles down to a sustained set of founder polymers and their direct and indirect reaction products.

Parallel experiments are carried out in which both the substrates and the catalysts are polypeptides. To do so, one may again begin with the minimal diversity 71 or 104 amino acid polypeptide libraries required to see the onset of catalysis of new molecular size products, then tune diversity upward several orders of magnitude. Minimally complex polypeptide systems can form a small number of novel product polymers which does not increase further over incubation time. A supracritical system shows an increasing diversity over time.

One-dimensional and two-dimensional gel electrophoresis are used to analyze the total increase in diversity over time. Unlike analysis of DNA sequences, however, use of two-dimensional gels may allow one to discriminate several novel product molecules with the same molecular weight on SDS page analysis. A sustained increase in total diversity over time (as limited by the product concentrations detectable), and a sustained increase in the highest molecular weight classes seen, is strong evidence for supracritical behavior of the reaction system.

In a second set of experiments, labeled amino acids and short peptides, up to hexamers, are incubated with libraries of increasing diversity from the larger amino acid library plus the polypeptide library. By one- and two-dimensional gel analysis, the labeled amino acids and small peptides are tested for incorporation into high molecular weight material. Control experiments use affinity purified ubiquitin alone with the labeled amino acids and small peptides, and the labeled amino acids and small peptides incubated by themselves.

Supracritical behavior may be demonstrated in a particularly clean way: Theoretical work shows that a sufficiently low diversity founder set of amino acids and small peptides will be subcritical. However, if the concentrations of members of that founder set are maintained by exogenous addition, and the set is incubated with a high diversity of larger polypeptides added once only at the outset of the experiment, then the larger polypeptides can catalyze the formation of many polypeptides built up out of the founder set. Those novel polypeptides themselves come to play catalytic roles in sustaining the formation of themselves and yet further novel polypeptides. Indeed, such a system might include collectively autocatalytic sets of polypeptides. In short, the small peptides alone, in sustained concentrations, are subcritical, but transient exposure to a high diversity of larger polypeptides triggers supracritical behavior which is thereafter sustained without further addition of the larger polypeptides.

To carry out this experiment, the above flow chemostat experiments are extended using labeled amino acids and small peptides, incubated with an initial set of diverse 71 or 104 amino acid polypeptides. The concentrations of the founder set of labeled amino acids and small peptides is sustained. At a critical diversity of 71 or 104 amino acid polypeptides, not only incorporation of amino acids and small peptides into high molecular weight material is seen, but persistence of that incorporation under the chemostat conditions which leads to the exponential dilution and ultimate loss of all initial 71 or 104 amino acid polypeptides. Such sustained synthesis of large polymers from the sustained founder set demonstrates that transient incubation with the high diversity library of 71 or 104 amino acid polypeptides triggers a phase transition in the system of amino acids and small peptides.

In order to confirm that exposure of a collection of organic molecules to a diversity of polypeptides leads to synthesis of an increasing diversity of organic molecules, a reliable means of detecting and discriminating small quantities of organic molecules is required. HPLC analysis appears to fulfill the requirements. With UV absorbance detection, HPLC can detect concentrations down to the nanomolar range. For example, tryptophan can be detected down to about 10 nanomolar. It may be possible to increase the range of small molecules which are detectable using IR rather than UV spectra (Kemp and Vellaccio, Organic Chemistry, Worth Publishers, Inc., 1980). A chosen set of fifty to a few hundred organic molecules gives rise to a discrete set of peaks which can be discriminated from a far more complex mixture containing a number of additional peaks due to the presence of new product molecules. Evidence of reactions include both the appearance of new peaks and the disappearance of the initial substrate peaks.

In these experiments, sets of founder organic molecules are first assembled with well-displaced peaks on HPLC analysis, followed by sequential addition of trial substrate compounds to solutions containing previously accepted members of the founder set. Founder sets are created which optimize both founder concentrations and diversity, such that novel product molecules yield easily detectable peaks.

As in the other experiments described above, experiments are carried out with a fixed input of founder organic molecules, and under conditions which drive forward synthesis and hold the system displaced from equilibrium by continuous addition of the founder set of organic molecules to otherwise closed stirred reaction systems. In a subset of experiments, radioactively labeled founder set molecules are used to establish that radioactive atoms are incorporated into new product molecules. The concentrations of product molecules ultimately depends upon the ratio of the diversity of founder set to product set, the number of reaction steps from the founder set to a given product molecule, and the detailed forward and reverse kinetics along the reaction pathway(s) leading to and from the product species. On average, however, if the founder set diversity is 100 and the set members are present in millimolar concentration initially, if the system were otherwise closed and if the final diversity were about ten million, then the terminal product concentrations might be about 10 nanomolar.

Once having established the conditions under which only a few reactions are catalyzed and thus in which product peaks are easily detected, the foundation is provided by which to increase the diversity of the polypeptides to which the same founder set is exposed. For a sufficient diversity of polypeptides, a very large increase in the diversity of small organic product molecules, hence peaks, is seen in the system. As in our analysis of systems using DNA or polypeptides of fixed initial length, here too, as reactions proceed, ever larger molecular weight products can be formed. Thus, in supracritical systems, both diversity and maximum molecular weight increase with time and with the diversity of the polypeptide library.

These experiments demonstrate that a large diversity of organic compounds can be formed by catalyzing reactions from a sustained founder set of small organic molecules. Thus, these experiments lead to the application of these new technologies to the generation of high diversity libraries of small molecules as drug candidates.

Once a diversity of novel organic products is generated, the logarithmically iterative procedure defined above may be utilized to isolate both the set of novel enzymes leading to a specific product molecule, and the set of founder organic molecules which are the initial substrates needed for the chain of reactions leading to the product molecule. This procedure is a minor modification of that described above and reflects the fact that several, e.g., 4, enzymes might be needed to catalyze a chain of reactions, and reflects the fact that several, e.g., 7, initial substrates may be required in those reactions. The four enzymes may be logarithmically isolated as follows. At each step, the current polypeptide library diversity is randomly partitioned into ten aliquots each containing a random 0.7 of the total diversity. The probability that any aliquot contains the four requisitive polypeptides is 0.24, hence on average two of the aliquots have the four enzymes. Reactions with the full diversity of initial substrates are carried out and the target of interest identified in one or two aliquots, thereby reducing the polypeptide library diversity by a factor 0.7. Successive cycles will, again in a logarithmic number of steps, isolate the four enzymes needed. To cut the substrate diversity down to the seven substrates needed, the substrate diversity is randomly assigned to 10 aliquots each containing a random 0.8 of the initial diversity. The probability that any aliquot has the seven critical substrates is 0.21, thus on average two aliquots are successful.

This analysis is of considerable interest for two reasons. First, it establishes that a sequence of reactions, not just a single reaction, is catalyzed by a set of novel enzymes, leading from a set of initial substrates in the founder collection to a target molecule many synthetic steps away. Second, such a procedure constitutes a radically new approach to the problems of organic synthesis. Here diversity and screening procedures are used to identify simultaneously not only de novo enzymes, but also the set of substrates leading via a sequence of catalyzed reactions to a target organic compound. The second interest, of course, relates to drug discovery.

There are several alternative approaches to finding such drug candidates. In a first, a receptor for the normal agonist is already in hand and is used to screen for small molecule mimics of the agonist. In a second, no receptor is yet available, but only the agonist itself. In a third, inhibitors of an enzyme are sought. As an example of the first approach, one might wish to detect the presence of an organic molecule of interest, present in nanomolar concentration, because it binds to a specific cloned cell receptor. Such detection is attainable by a competition assay with the normal ligand for the cloned receptor. Labeled normal ligand would not bind or would show reduced binding in the presence of the entirely unknown small molecule present in the reaction mixture. As discussed below, nanomolar concentrations suffice for detection. Where a binding event is detected when the unknown product is in the nanomolar range, then the above described logarithmic dilution process may be used to find both the enzymes and substrates leading to synthesis of a new organic molecule able to bind a cell receptor. Note that neither the target molecule, nor the specific initial substrates, nor the enzymes required for synthesis of the target from the founder set of substrates, need to be known in advance. Any such molecule is a drug candidate to bind to the receptor, hence modify or mimic or antagonize the activity of the normal agonist.

In the second approach, the receptor for the agonist is not known, but the agonist is known. Here a set of random polypeptides which bind to the agonist, hence are its shape complements, is sought. This set of polypeptides then can be used, in place of the unknown receptor, to screen for novel organic molecules which compete with the agonist for binding to members of the set of shape complement polypeptides. While one would not yet know which polypeptides bound the agonist by groups of atoms which reflected the function of the agonist, some among the polypeptides presumably do bind the important agonist epitopes. Thus, the set of organic molecules binding to the polypeptide set is a set of candidate drugs to mimic or modulate the activity of the agonist.

A third approach seeks a novel small molecule inhibitor of an enzyme such as HIV protease by slowing cleavage of the peptide substrate.

To seek agonist mimetics of estrogen, for example, the cloned estrogen receptor which is immobilized on Immobilon P filters as dot blot arrays is utilized. Competition assays are carried out with radioactively labeled estrogen and the molecules formed in the reaction mixtures. Dot blot filters are incubated with decreasing concentrations of labeled estrogen and constant concentrations of the mixture of organic molecules. Control filters have no organic molecules added. As estrogen concentration decreases, tests are conducted to determine whether competitive displacement of the labeled estrogen occurs. Tritium labeled estrogen and its analogues are available as 150 Ci per millimole. Thus, a picomole of this probe is 0.15 microcuries. ¹²⁵I labeled estrogen and its analogues labeled at over 2200 Ci per millimole are available. A picomole is 2.2 microcuries. Thus, even less than picomole quantities of organic molecule competitors which displace such bound labeled estrogen are detectable. Since novel products in the 100 to 1000 picomolar range are generated, even estrogen mimics with modest affinity for the receptor displace labeled estrogen present in picomole concentration, and thus are detectable.

From the foregoing, it will be appreciated that, although specific embodiments of the invention have been described herein for purposes of illustration, various modifications may be made without deviating from the spirit and scope of the invention.

1-50. (Canceled).

51. A method for the production of an organic molecule having a desired property, comprising the steps of:
(a) providing a reaction mixture with at least 10 different organic molecules in solution in the same reaction container;
(b) causing at least one random chemical reaction to take place with at least some of the different organic molecules in the reaction mixture to create a reaction mixture having one or more organic molecules different from the organic molecules in the starting group of the previous step;
(c) repeating step (b) at least once by causing at least one random chemical reaction to take place with at least some of the organic molecules in the reaction mixture from the previous step or repetition to thereby produce a final reaction mixture as a result of the last repetition; and
(d) screening the final reaction mixture resulting from step (c) for the presence of the organic molecule having the desired property.
